(19) United States
(12) Patent Application Publication
(10) Pub. No.: US 2026/0103668 A1
(43) Pub. Date: Apr. 16, 2026

(54) SYSTEMS FOR UV-STERILIZING BIOPROCESS INTERCONNECTIONS AND FOR UV-STERILIZING BIOPROCESSING MEDIA

(71) Applicant: ARCOLOGY INC. DBA BIOSPHERE, Culver City, CA (US)

(72) Inventors: Anthony ABEL, Berkeley, CA (US); Brian HELIGMAN, North Potomac, MD (US)

(21) Appl. No.: 19/353,500

(22) Filed: Oct. 8, 2025

Related U.S. Application Data
(60) Provisional application No. 63/705,873, filed on Oct. 10, 2024.

(51) Int. Cl. C12M 1/00; C12M 1/12

(52) U.S. Cl. CPC C12M 29/26; C12M 23/22; C12M 29/06; C12M 37/00

(57) ABSTRACT

The disclosed technology provides an alternative means of efficient sterilization when transferring sterile media or axenic culture, or when sterilizing media for a bioprocess. Some variations provide a process interconnection comprising: a first process valve; a second process valve in flow communication with the first process valve; a process chamber in flow communication, and interposed between, the first and second process valves; and one or more UV light sources configured to expose ultraviolet light to at least 99% of chamber internal surface area and to at least 99% of valve internal surface area. Some variations provide a system for bioprocessing that utilizes the process interconnection. Other variations provide a system for sterilizing bioprocessing media. Various methods of using the disclosed systems are also disclosed in detail. These include methods of sterile material transfer, methods of axenic culture transfer, methods of sterile bioprocessing, and methods of sterilizing bioprocessing media.

### PRIORITY DATA

This non-provisional patent application claims priority to U.S. Provisional Patent App. No. 63/705,873, filed on Oct. 10, 2024, which is hereby incorporated by reference.

### FIELD

The present invention generally relates to sterilization within bioprocesses. More particularly, the present invention relates to the design of bioprocess interconnections and other bioprocess components for efficient sterilization, and methods of sterilizing bioprocess interconnections and other bioprocess components.

### BACKGROUND

Biological production of products typically requires the elimination to the greatest extent possible of contaminating microorganisms, otherwise known as adventitious agents or non-host contaminants. Contamination can cause conversion of nutrients into undesirable products, change the conditions of the broth, or degrade or otherwise adversely affect the product(s). In modern bioprocess facilities, the presence of adventitious agents is reduced by thorough cleaning, most commonly by steam. This process is referred to as sterilization. In some cases, a small fraction of the contaminating organisms remains active within the system.

In bioprocess facilities, the sterile transfer of fluids from one component or piece of equipment to another in a process is highly desirable. Air or other gaseous materials are frequently supplied to modern bioreactors to enable, for example, aerobic fermentation. Sterile liquid media is supplied to a sterile bioreactor in advance of a fermentation process, or during a fermentation process to enable further growth and product formation. Culture is also often transferred from a seed bioreactor to a production bioreactor. Such transfers can be a major source of contamination in bioprocesses.

See Junker et al., “Sustainable Reduction of Bioreactor Contamination in an Industrial Fermentation Pilot Plant”, Journal of Bioscience and Bioengineering, Vol. 102, No. 4, Pages 251-268, 2006 (hereinafter, “Junker”), which is hereby incorporated by reference. According to Junker, contaminated media transfer is a commonly suspected cause of contamination in industrial bioprocessing facilities.

Steam sterilization is widely utilized in bioprocess facilities. However, steam sterilization suffers from several problems. Steam by nature is dangerous and can cause severe burns to a human if there is a steam leak. Steam sterilization process failures are well-known and can be caused not only by human errors, but also by steam quality problems, such as incorrect temperature or pressure, insufficient air removal from steam, and so on. Steam sterilization process failures can also be caused by incorrect design or placement of process components to be sterilized. In some cases, steam is unable to sterilize a certain process component due to very small cracks (harboring contaminant microorganisms) where those cracks are hard to find. There are known cases where process surfaces are physically blocked by valves when steam sterilization is running, where the process surfaces become unblocked when the process is operating, leading to mysterious contamination problems.

In addition, repeated sterilizations cycles can cause undesirable changes in the material properties of process piping and components. Possible changes include corrosion that alters the chemical composition of the metal or polymer; and erosion, pipe thinning, surface pitting, or phase changes that alter the mechanical properties of the metal or polymer. One example in the case of polymers is embrittlement caused by repeated sterilizations cycles. For both metals and polymers, steam leakage or even catastrophic failures of process piping and components can occur. Even stainless steel is not immune to problems from repeated sterilizations cycles. During a sterilization cycle, there is heating, sterilizing and cooling. During each of these phases, alloy stresses or metal oxidation can result from temperature and pressure changes. Steam corrosion in stainless steel can be reduced with a corrosion inhibitor, such as cyclohexylamine, but usually such additives cannot be used in a bioprocess for safety reasons.

Steam sterilization is also energy-intensive. Conventional autoclaves utilize steam generated by burning natural gas, resulting in non-renewable CO₂ emissions and therefore adding to the overall carbon intensity of the particular bioprocess.

In view of the need for sterilization during bioprocessing, and the challenges posed by steam sterilization, there is a tremendous need for an alternative means of sterilization when transferring sterile media or axenic culture, or when sterilizing media in a bioprocess.

### SUMMARY

The present invention addresses the aforementioned needs in the art, as will now be summarized and then further described in detail below.

Some variations of the disclosed technology provide systems.

Some variations provide a process interconnection comprising:
(a) a first process valve comprising a first outer port and a first inner port;
(b) a second process valve comprising a second outer port and a second inner port, wherein the second process valve is in flow communication with the first process valve;
(c) a process chamber interposed between the first inner port and the second inner port, wherein the process chamber is in flow communication with the first process valve and with the second process valve, and wherein the process chamber has internal chamber surfaces; and
(d) one or more UV light sources configured to (i) expose ultraviolet light to at least 99% of chamber internal surface area defined by total surface area of the internal chamber surfaces and (ii) expose the ultraviolet light to at least 99% of valve internal surface area defined by total surface area of all internal, wettable valve surfaces within the first process valve and the second process valve.

In some embodiments, the one or more UV light sources are configured to expose ultraviolet light to 100% of the chamber internal surface area and/or to 100% of the valve internal surface area.

In some embodiments, the first process valve, the second process valve, and the process chamber are fabricated from materials that are substantially chemically inert to the ultraviolet light.

In some embodiments, the process interconnection further comprises a third process valve having a third outer port and a third inner port, wherein the third process valve is in flow communication with the process chamber, the first process valve, and the second process valve.

In some embodiments, the process interconnection further comprises a third process valve having a third outer port and a third inner port, as well as a fourth process valve having a fourth outer port and a fourth inner port, wherein the third process valve is in flow communication with the process chamber, wherein the fourth process valve is in flow communication with the process chamber, and wherein the third process valve is in flow communication with the fourth process valve.

In some embodiments, the first inner port is connected to the process chamber using a first sanitary connection, and the second inner port is connected to the process chamber using a second sanitary connection.

In some embodiments, the first inner port is connected to the process chamber using a first reversible sanitary connection, and the second inner port is connected to the process chamber using a second reversible sanitary connection.

In some embodiments, the one or more UV light sources are positioned externally to the process chamber, the first process valve, and the second process valve. In these embodiments, the process chamber is configured with chamber walls that are substantially transparent or translucent to the ultraviolet light. Optionally, the first process valve and the second process valve are configured with valve walls that are substantially transparent or translucent to the ultraviolet light.

In some embodiments, the one or more UV light sources are positioned internally within the process chamber. Optionally, the one or more UV light sources are located inside a sleeve that is positioned inside the process chamber, wherein the sleeve is substantially transparent or translucent to the ultraviolet light.

In some embodiments, the process chamber is a vessel configured to contain a bioprocessing material. The process chamber may be configured with one or more additional sanitary ports.

Other variations provide a system for bioprocessing, the system comprising:
(a) a bioreactor comprising a bioreactor chamber configured to carry out a reaction;
(b) a bioreactor port configured for introducing bioreaction media to the bioreactor chamber;
(c) a first process valve comprising a first outer port and a first inner port, wherein the first process valve is in flow communication with the bioreactor port;
(d) a process chamber interposed between the first inner port and the bioreactor port, wherein the process chamber is in flow communication with the first process valve and with the bioreactor port, and wherein the process chamber has internal chamber surfaces; and
(e) one or more UV light sources configured to (i) expose ultraviolet light to at least 99% of chamber internal surface area defined by total surface area of the internal chamber surfaces and (ii) expose the ultraviolet light to at least 99% of first-valve internal surface area defined by total surface area of all internal, wettable valve surfaces within the first process valve.

In some embodiments, the one or more UV light sources are configured to expose ultraviolet light to 100% of the chamber internal surface area and/or to 100% of the first-valve internal surface area.

In some embodiments, the first process valve and the process chamber are fabricated from materials that are substantially chemically inert to the ultraviolet light.

In some embodiments, the bioreactor port is a sanitary bioreactor port.

In some embodiments, the system further comprises a second process valve interposed between the process chamber and the bioreactor port, wherein the second process valve comprises a second outer port and a second inner port, and wherein the second process valve is in flow communication with the first process valve and with the bioreactor chamber. Optionally, the one or more UV light sources may be configured to expose the ultraviolet light to at least 99% of second-valve internal surface area defined by total surface area of all internal, wettable valve surfaces within the second process valve.

In some embodiments, the system further comprises a third process valve having a third outer port and a third inner port, wherein the third process valve is in flow communication with the process chamber, the first process valve, and the second process valve.

In some embodiments, the bioreactor comprises a component configured for introducing a gas into the bioreactor chamber.

In some embodiments, the bioreactor comprises one or more bioreactor UV light sources configured to expose additional ultraviolet light to surfaces within the bioreactor chamber.

In some embodiments, the one or more UV light sources are positioned externally to the process chamber and the first process valve, wherein the process chamber is configured with chamber walls that are substantially transparent or translucent to the ultraviolet light.

Optionally, the first process valve is configured with valve walls that are substantially transparent or translucent to the ultraviolet light.

In some embodiments, the one or more UV light sources are positioned internally within the process chamber.

Other variations provide a system for sterilizing bioprocessing media, the system comprising:
(a) a vessel configured for containing bioprocessing media;
(b) a vessel port configured for introducing the bioprocessing media to the vessel and/or for withdrawing the bioprocessing media from the vessel;
(c) a first process valve comprising a first outer port and a first inner port, wherein the first process valve is in flow communication with the vessel port;
(d) a process chamber in flow communication with the first process valve and the vessel port, wherein the process chamber has internal chamber surfaces and a process-chamber flow path through which the bioprocessing media must pass for the bioprocessing media to be introduced to and/or withdrawn from the vessel; and
(e) one or more UV light sources configured to expose ultraviolet light to at least 99% of the process-chamber flow path.

In some embodiments, the one or more UV light sources are configured to expose the ultraviolet light to at least 99% of chamber internal surface area defined by total surface area of the internal chamber surfaces.

In some embodiments, the one or more UV light sources are configured to expose the ultraviolet light to at least 99% of first-valve internal surface area defined by total surface area of all internal, wettable valve surfaces within the first process valve.

In certain embodiments, the one or more UV light sources are configured to expose the ultraviolet light to at least 99% of chamber internal surface area defined by total surface area of the internal chamber surfaces, and to expose the ultraviolet light to at least 99% of first-valve internal surface area defined by total surface area of all internal, wettable valve surfaces within the first process valve.

In some embodiments, the vessel port is a sanitary vessel port.

In some embodiments, the process chamber is interposed between the first inner port and the vessel port.

In some embodiments, the system further comprises a second process valve interposed between the process chamber and the vessel port, wherein the second process valve comprises a second outer port and a second inner port, and wherein the second process valve is in flow communication with the first process valve and with the vessel. Optionally, the one or more UV light sources are optionally configured to expose the ultraviolet light to at least 99% of second-valve internal surface area defined by total surface area of all internal, wettable valve surfaces within the second process valve.

In some embodiments, the process chamber is in flow communication with the first outer port, and the process chamber is not interposed between the first inner port and the vessel port.

In some embodiments, the vessel is a bioreactor comprising a bioreactor chamber configured to carry out a reaction involving the bioprocessing media.

In some embodiments, the vessel is a tank configured to contain the bioprocessing media.

In some embodiments, a filtration system is positioned in flow communication with the first process valve. In these or other embodiments, an in-line sonicator is positioned in flow communication with the first process valve.

In some embodiments, the one or more UV light sources are positioned externally to the process chamber and the first process valve, wherein the process chamber is configured with chamber walls that are substantially transparent or translucent to the ultraviolet light.

Optionally, the first process valve is configured with valve walls that are substantially transparent or translucent to the ultraviolet light.

In some embodiments, the one or more UV light sources are positioned internally within the process chamber.

Some variations of the disclosed technology provide methods.

In some variations, a method of sterile material transfer comprises:
(i) providing a process interconnection comprising: (a) a first process valve comprising a first outer port and a first inner port; (b) a second process valve comprising a second outer port and a second inner port, wherein the second process valve is in flow communication with the first process valve; (c) a process chamber interposed between the first inner port and the second inner port, wherein the process chamber is in flow communication with the first process valve and with the second process valve, and wherein the process chamber has internal chamber surfaces; and (d) one or more UV light sources configured to expose ultraviolet light to the internal chamber surfaces and to all internal, wettable valve surfaces within the first process valve and the second process valve;
(ii) providing a first sterile container that contains a sterile material to be transferred and a second sterile container that receives the sterile material, wherein the first sterile container is connected to the first outer port, and wherein the second sterile container is connected to the second outer port;
(iii) ensuring the first process valve and the second process valve are each in a closed position;
(iv) activating the one or more UV light sources to deliver the ultraviolet light to at least 99% of chamber internal surface area defined by total surface area of the internal chamber surfaces and to at least 99% of valve internal surface area defined by total surface area of all internal, wettable valve surfaces within the first process valve and the second process valve, using a UV dose effective to substantially deactivate adventitious microorganisms from the internal chamber surfaces and from the internal, wettable valve surfaces within the first process valve and the second process valve;
(v) opening the first process valve and the second process valve;
(vi) transferring the sterile material from the first sterile container to the second sterile container, through the process chamber; and
(vii) closing the first process valve and the second process valve.

In some method embodiments, the one or more UV light sources expose the ultraviolet light to 100% of the chamber internal surface area.

In some method embodiments, the one or more UV light sources expose the ultraviolet light to 100% of the valve internal surface area.

In certain method embodiments, the one or more UV light sources expose the ultraviolet light to 100% of the chamber internal surface area and 100% of the valve internal surface area.

In some method embodiments, the sterile material is selected from the group consisting of a sugar, a salt, a vitamin, a mineral, a pH buffer, an antifoam, an alcohol, an acid, a base, water, air, carbon dioxide, carbon monoxide, syngas, methane, hydrogen, nitrogen, argon, ammonia, a solution of any one of the foregoing, and combinations thereof.

In some method embodiments, the first sterile container is a sterile holding tank, the second sterile container is a reactor, and the method transfers sterile media from the sterile holding tank into the reactor.

Preferably, step (iv) is effective to reach at least a 6-log reduction in the adventitious microorganisms.

Other variations provide a method of axenic culture transfer, the method comprising:
(i) providing a process interconnection comprising: (a) a first process valve comprising a first outer port and a first inner port; (b) a second process valve comprising a second outer port and a second inner port, wherein the second process valve is in flow communication with the first process valve; (c) a process chamber interposed between the first inner port and the second inner port, wherein the process chamber is in flow communication with the first process valve and with the second process valve, and wherein the process chamber has internal chamber surfaces; and (d) one or more UV light sources configured to expose ultraviolet light to the internal chamber surfaces and to all internal, wettable valve surfaces within the first process valve and the second process valve;
(ii) providing a first container that contains an axenic culture to be transferred and a second container that receives the axenic culture, wherein the first container is connected to the first outer port, wherein the second container is connected to the second outer port, and wherein the second container either is sterile prior to receiving the axenic culture, or contains an initial quantity of the axenic culture;
(iii) ensuring the first process valve and the second process valve are each in a closed position;
(iv) activating the one or more UV light sources to deliver the ultraviolet light to at least 99% of chamber internal surface area defined by total surface area of the internal chamber surfaces and to at least 99% of valve internal surface area defined by total surface area of all internal, wettable valve surfaces within the first process valve and the second process valve, using a UV dose effective to substantially deactivate adventitious microorganisms from the internal chamber surfaces and from the internal, wettable valve surfaces within the first process valve and the second process valve;
(v) opening the first process valve and the second process valve;
(vi) transferring the axenic culture from the first container to the second container, through the process chamber; and
(vii) closing the first process valve and the second process valve.

In some method embodiments, the second container is sterile prior to receiving the axenic culture. In other embodiments, the second container is not sterile prior to receiving the axenic culture, but contains an initial quantity of the axenic culture and no other microorganisms.

In some method embodiments, the first container is a first axenic reactor, the second container is a second axenic reactor, and the method transfers the axenic culture from the first axenic reactor into the second axenic reactor.

In some method embodiments, the first container is an axenic reactor, the second container is a reactor cooling loop, the method transfers reactor contents from the axenic reactor into the reactor cooling loop, and the method further transfers cooled reactor contents from the reactor cooling loop back to the axenic reactor.

The axenic culture may be selected from bacteria, yeasts, and filamentous fungi, for example.

Other variations provide a method of sterile bioprocessing, the method comprising:
(i) providing a bioreactor comprising a bioreactor chamber configured to carry out a reaction, wherein the bioreactor is configured with a bioreactor port for introducing bioreaction media to the bioreactor chamber;
(ii) providing a process interconnection comprising: (a) a first process valve comprising a first outer port and a first inner port, wherein the first process valve is in flow communication with the bioreactor port; (b) a process chamber interposed between the first inner port and the bioreactor port, wherein the process chamber is in flow communication with the first process valve and with the bioreactor port, and wherein the process chamber has internal chamber surfaces; and (c) one or more UV light sources configured to expose ultraviolet light to the internal chamber surfaces and to all internal, wettable valve surfaces within the first process valve;
(iii) operating the bioreactor containing the bioreaction media to carry out the reaction, to convert one or more substrates into one or more products; and
(iv) before, during, and/or after step (iii), activating the one or more UV light sources to expose the ultraviolet light to at least 99% of chamber internal surface area defined by total surface area of the internal chamber surfaces and at least 99% of first-valve internal surface area defined by total surface area of all internal, wettable valve surfaces within the first process valve, using a UV dose effective to substantially deactivate adventitious microorganisms, contained within the bioreaction media, from the internal chamber surfaces and from the first-valve internal surface area.

In some method embodiments, the one or more UV light sources are configured to expose ultraviolet light to 100% of the chamber internal surface area and/or to 100% of the first-valve surface area.

In some method embodiments, the bioreactor port is a sanitary bioreactor port.

In some method embodiments, the process interconnection further comprises a second process valve interposed between the process chamber and the bioreactor port; wherein the second process valve comprises a second outer port and a second inner port; wherein the second process valve is in flow communication with the first process valve and with the bioreactor chamber; wherein during step (iv), the one or more UV light sources expose the ultraviolet light to at least 99% of second-valve internal surface area defined by total surface area of all internal, wettable valve surfaces within the second process valve; and wherein the UV dose is effective to substantially deactivate adventitious microorganisms from the second-valve internal surface area.

In some method embodiments, the process interconnection further comprises a third process valve having a third outer port and a third inner port, wherein the third process valve is in flow communication with the process chamber, the first process valve, and the second process valve.

In some method embodiments, during step (iv), the first process valve is closed. In other embodiments, during step (iv), the first process valve is open.

In some method embodiments, the method further comprises pre-filtering the bioreaction media prior to exposing the bioreaction media to the ultraviolet light.

In some method embodiments, the method further comprises sonicating the bioreaction media prior to exposing the bioreaction media to the ultraviolet light.

In preferred method embodiments, step (iv) is effective to reach a 6-log reduction in the adventitious microorganisms within the process chamber and the first process valve.

Still other variations provide a method of sterilizing a bioprocessing media, the method comprising:
(i) providing a vessel configured for containing bioprocessing media, wherein the vessel is configured with a vessel port for introducing the bioprocessing media to the vessel and/or for withdrawing the bioprocessing media from the vessel;
(ii) providing a process interconnection comprising: (a) a first process valve comprising a first outer port and a first inner port, wherein the first process valve is in flow communication with the vessel port; (b) a process chamber in flow communication with the first process valve and the vessel port, wherein the process chamber has internal chamber surfaces and a process-chamber flow path through which the bioprocessing media must pass for the bioprocessing media to be introduced to and/or withdrawn from the vessel; and (c) one or more UV light sources configured to expose ultraviolet light to the internal chamber surfaces;
(iii) adding the bioprocessing media to the vessel, from a media source vessel; or withdrawing the bioprocessing media from the vessel, to a media receiving vessel; and
(iv) during step (iii), activating the one or more UV light sources to expose the ultraviolet light to at least 99% of the process-chamber flow path, using a UV dose effective to substantially deactivate adventitious microorganisms contained within the bioprocessing media, thereby generating a sterilized bioprocessing media,
wherein step (iv) is effective to reach a 6-log reduction in the adventitious microorganisms within the process chamber.

In some method embodiments, the one or more UV light sources are configured to expose the ultraviolet light to at least 99% of chamber internal surface area defined by total surface area of the internal chamber surfaces.

In some method embodiments, the one or more UV light sources are configured to expose the ultraviolet light to at least 99% of first-valve internal surface area defined by total surface area of all internal, wettable valve surfaces within the first process valve.

In certain method embodiments, the one or more UV light sources are configured to expose the ultraviolet light to at least 99% of chamber internal surface area defined by total surface area of the internal chamber surfaces as well as at least 99% of first-valve internal surface area defined by total surface area of all internal, wettable valve surfaces within the first process valve.

In some method embodiments, the vessel port is a sanitary vessel port.

In some method embodiments, the process interconnection further comprises a second process valve interposed between the process chamber and the vessel port; wherein the second process valve comprises a second outer port and a second inner port; and wherein the second process valve is in flow communication with the first process valve and with the vessel. In some method embodiments in which there is a second process valve, during step (iv), the one or more UV light sources expose the ultraviolet light to at least 99% of second-valve internal surface area defined by total surface area of all internal, wettable valve surfaces within the second process valve; and wherein the UV dose is effective to substantially deactivate adventitious microorganisms from the second-valve internal surface area.

In some method embodiments, the bioprocessing media is selected from the group consisting of a sugar, a salt, a vitamin, a mineral, a pH buffer, an antifoam, an alcohol, an acid, a base, water, air, carbon dioxide, carbon monoxide, syngas, methane, hydrogen, nitrogen, argon, ammonia, a solution of any one of the foregoing, and combinations thereof.

In some method embodiments, the vessel is a bioreactor comprising a bioreactor chamber configured to carry out a reaction involving the sterilized bioprocessing media, and the method transfers sterilized bioprocessing media into the bioreactor through the vessel port.

In some method embodiments, the vessel is a bioreactor comprising a bioreactor chamber configured to carry out a reaction involving the sterilized bioprocessing media, and the method transfers sterilized bioprocessing media out of the bioreactor through the vessel port.

In some method embodiments, step (iii) comprises adding the bioprocessing media to the vessel, from the media source vessel; and the vessel is a tank configured to contain the sterilized bioprocessing media.

In some method embodiments, step (iii) comprises withdrawing the bioprocessing media from the vessel, into the media receiving vessel; and the vessel is a tank configured to contain the bioprocessing media prior to being exposed to the ultraviolet light.

In some method embodiments, step (iii) comprises withdrawing the bioprocessing media from the vessel; and the media receiving vessel is the environment.

In some embodiments, the method further comprises pre-filtering the bioprocessing media prior to exposing the bioprocessing media to the ultraviolet light.

In some embodiments, the method further comprises sonicating the bioprocessing media prior to exposing the bioprocessing media to the ultraviolet light.

### BRIEF DESCRIPTION OF THE DRAWINGS

FIG. 1A (side view) depicts an exemplary process interconnection comprising a first process valve, a second process valve, a process chamber interposed between the first process valve and the second process valve, and a UV light source configured to expose ultraviolet light to internal surfaces within the process chamber and preferably configured to expose ultraviolet light to internal surfaces within the first and second process valves.

FIG. 1B (side view) depicts an exemplary process interconnection comprising a first process valve with a first outer port and a first inner port; a second process valve with a second outer port and a second inner port, wherein the second process valve is in flow communication with the first process valve; a process chamber interposed between the first inner port and the second inner port, wherein the process chamber has internal chamber surfaces; and a UV light source configured to expose ultraviolet light to the internal chamber surfaces.

FIG. 1C (isometric 3D view) depicts an exemplary process interconnection comprising a first process valve with a first outer port and a first inner port; a second process valve with a second outer port and a second inner port, wherein the second process valve is in flow communication with the first process valve; a process chamber interposed between the first inner port and the second inner port, wherein the process chamber has internal chamber surfaces; and a UV light source configured to expose ultraviolet light to the internal chamber surfaces.

FIG. 2A (side view) depicts an exemplary process interconnection comprising a first process valve, a second process valve, a third process valve, a fourth process valve, a process chamber interposed between the first process valve and the second process valve and interposed between the third process valve and the fourth process valve, a first UV light source configured to expose ultraviolet light to internal surfaces within the process chamber and within the first process valve, and a second UV light source configured to expose ultraviolet light to internal surfaces within the process chamber and within the second process valve.

FIG. 2B (side view) depicts an exemplary process interconnection comprising a first process valve, a second process valve, a third process valve, a fourth process valve, multiple process chambers, a first UV light source configured to expose ultraviolet light to internal surfaces within a first process chamber and within the first process valve, a second UV light source configured to expose ultraviolet light to internal surfaces within a second process chamber and within the second process valve, a third UV light source configured to expose ultraviolet light to internal surfaces within a third process chamber and within the third process valve, a fourth UV light source configured to expose ultraviolet light to internal surfaces within a fourth process chamber and within the fourth process valve, and optionally a fifth UV light source configured to expose ultraviolet light to internal surfaces within a fifth process chamber.

FIG. 2C (isometric 3D view) depicts an exemplary process interconnection comprising a first process valve, a second process valve, a third process valve, a fourth process valve, multiple process chambers, a first UV light source configured to expose ultraviolet light to internal surfaces within a first process chamber and within the first process valve, a second UV light source configured to expose ultraviolet light to internal surfaces within a second process chamber and within the second process valve, a third UV light source configured to expose ultraviolet light to internal surfaces within a third process chamber and within the third process valve, a fourth UV light source configured to expose ultraviolet light to internal surfaces within a fourth process chamber and within the fourth process valve, and optionally a fifth UV light source configured to expose ultraviolet light to internal surfaces within a fifth process chamber.

FIG. 3 (side view) depicts an exemplary process interconnection comprising a first process valve, a second process valve, a process chamber configured with a UV light source to expose ultraviolet light to internal surfaces within the process chamber, a bioreactor equipped with a bioreactor sparger and a bioreactor valve, and process piping connecting the first process valve to a second process valve in fluid communication with the bioreactor.

FIG. 4 (side view) depicts an exemplary process interconnection comprising a first process valve, a second process valve, a process chamber interposed between the first process valve and the second process valve, and a UV light source configured to expose ultraviolet light to internal surfaces within the process chamber, wherein the second process valve is directly connected to a bioreactor equipped with a bioreactor sparger and a bioreactor valve.

FIG. 5A (side view) depicts an exemplary system comprising a bioreactor with a bioreactor sparger and a bioreactor valve, a first process valve, a second process valve, a process chamber interposed between the first process valve and the second process valve, and a UV light source configured to expose ultraviolet light to internal surfaces within the process chamber, wherein the second process valve is directly connected to the bioreactor.

FIG. 5B (side view) depicts an exemplary system comprising a bioreactor with a bioreactor sparger and a bioreactor valve, a first process valve, a sanitary port (no valve), a process chamber interposed between the first process valve and the sanitary port, and a UV light source configured to expose ultraviolet light to internal surfaces within the process chamber, wherein the sanitary port is directly connected to the bioreactor.

### DETAILED DESCRIPTION OF EMBODIMENTS

The systems and methods of the present invention will be described in detail by reference to various non-limiting embodiments.

This description will enable one skilled in the art to make and use the invention, and it describes several embodiments, adaptations, variations, alternatives, and uses of the invention. These and other embodiments, features, and advantages of the present invention will become more apparent to those skilled in the art when taken with reference to the following detailed description of the invention in conjunction with any accompanying figures.

As used in this specification and the appended claims, the singular forms “a,” “an,” and “the” include plural referents unless the context clearly indicates otherwise. Unless defined otherwise, all technical and scientific terms used herein have the same meaning as is commonly understood by one of ordinary skill in the art to which this invention belongs.

Unless otherwise indicated, all numbers expressing conditions, concentrations, dimensions, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phrase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phrase “consisting essentially of” limits the scope of a claim to the specified elements or method steps, plus those that do not materially affect the basis and novel characteristic(s) of the claimed subject matter.

With respect to the terms “comprising,” “consisting of,” and “consisting essentially of,” where one of these three terms is used herein, the presently disclosed and claimed subject matter may include the use of either of the other two terms, except when used in a Markush group. Thus in some embodiments not otherwise explicitly recited, any instance of “comprising” may be replaced by “consisting of” or, alternatively, by “consisting essentially of.”

The present invention, in some variations, is predicated on the use of ultraviolet (UV) light to sterilize process interconnections, enabling the sterile transfer of media or other materials from one process location to another process location, or the sterilization of a material prior to being conveyed to a process location. A “process interconnection” (also referred to as a “process interconnect” or a “UV interconnect”) is a physical apparatus.

In some variations, a UV interconnect consists of a first process valve, a process chamber, at least one additional process valve, and one or more UV light sources. The UV light source may be inside the process chamber or outside the process chamber. In preferred embodiments, the UV light source is capable of directing UV light to all wetted surfaces of the process valves and process chamber. In preferred embodiments, the wetted (or wettable) surfaces and wetted (or wettable) seals have surfaces (e.g., with low surface roughness) that are capable of being effectively sterilized by UV radiation. In some embodiments, the materials of construction for the process valves and process chamber are designed to be substantially inert to UV radiation.

The process valves within the UV interconnect may be any type of process valve, depending on the requirements of the process. For example, process valves may be selected from butterfly valves, diaphragm valves, ball valves, globe valves, check-style valves, or other types of known valves.

In some embodiments, a UV interconnect is used to connect a process or storage vessel and a process line. In some embodiments, a UV interconnect is used to connect two process lines. In some embodiments, a UV interconnect is used to connect a first process or storage vessel to a second process or storage vessel. In some embodiments, UV interconnects are used to connect first process or storage vessels or lines to second process or storage vessels or lines.

In some embodiments, a UV interconnect is permanently attached to process piping, such as via welding or joining. In some embodiments, a UV interconnect is attached to process piping in a reversible manner to enable process flexibility, such as via the use of a clamp or flange-style connection.

The “process chamber” should be broadly construed herein. In some embodiments, no chemistry or biochemistry occurs within the process chamber. In other embodiments, chemistry or biochemistry occurs within the process chamber, in which case the process chamber may also be referred to as a reaction chamber.

In some embodiments, the process chamber is an interior region of a tube that minimizes flow obstruction. The process chamber may be characterized by a wide range of chamber volume or chamber length, as is desirable for the process.

In certain embodiments, the process chamber includes various process elements that are desirable to accomplish heat transfer (heating or cooling), mass transfer (e.g., mixing), chemical or biological reactions, or a combination thereof. Process elements within the process chamber may include static mixing elements or heat-exchange elements, for example.

In some embodiments, all flow volume between the first inner port and the second inner port constitutes the process chamber. In other embodiments, in addition to a process chamber, there are additional sections of pipe, mixers, sensors, flow controllers, additional valves, and so on. In preferred embodiments, the process chamber is configured to maintain a sterile boundary with the environment. A sterile boundary means that adventitious microorganisms do not penetrate into the process chamber from the environment.

The process chamber is preferably designed such that the UV dose exposed to the wettable surfaces, delivered by the UV light source(s), is sufficient to accomplish the desired reduction of the contaminant (e.g., adventitious microorganisms) suitable for the desired process, in a reasonable amount of time. For example, if it is determined that a UV dose of 300 mJ/cm² is desirable to substantially eliminate all adventitious microorganisms, this UV dose may be accomplished by providing a UV light intensity at a target wavelength of at least 0.1 mW/cm² to all wettable surfaces for at least 3000 seconds, or a light intensity of 1 mW/cm² to all wettable surfaces for at least 300 seconds, or a light intensity of at least 10 mW/cm² for at least 30 seconds, or any other combination of UV light intensity and UV time sufficient to achieve the desired UV dose. The units of mW/cm² are milliwatts UV light power divided by process-chamber wettable surface area in square centimeters.

In some embodiments, a UV light source is located inside a sleeve, which is in turn positioned inside the process chamber of the UV interconnect. The sleeve is used to protect the UV light source(s) from the process material(s) during operation. In these embodiments, the sleeve is preferably made from a sleeve material that is substantially transparent or translucent to the UV radiation delivered by the UV light source. In some embodiments, the sleeve itself is exposed to process material in the process chamber, so the sleeve material is preferably of sufficient surface quality so that the sleeve is sterilizable by UV radiation.

In some embodiments, a UV light source is positioned outside the process chamber. In these embodiments, the process chamber is preferably fabricated with a chamber wall material that is substantially transparent or translucent to sterilizing UV radiation.

The present invention, in various embodiments, is characterized by a number of important advantages.
1. The disclosed UV interconnect avoids thermal stress on process connections that, over time, ordinarily cause corrosion, pitting, cracking, wear, and buildup of contaminants.
2. The disclosed UV interconnect enables effectively complete sterilization in preparation for process fluid transfer—or during transfer in the case of media sterilization—which has heretofore not been achievable.
3. The disclosed UV interconnect enables the use of alternative materials that are not conducive to traditional steam-based sterilization methods. The disclosed UV interconnect may utilize thermoplastics that would soften, melt, or decompose if exposed to sterilizing steam or heat.
4. The disclosed UV interconnect reduces the cost and time required to sterilize process connections compared to traditional methods, by (i) eliminating the need for additional piping to deliver steam to a process connection, (ii) eliminating the need for steam traps in process systems, and/or (iii) reducing the contact time required for sterilization.

Some variations provide a process interconnection comprising:
(a) a first process valve comprising a first outer port and a first inner port;
(b) a second process valve comprising a second outer port and a second inner port, wherein the second process valve is in flow communication with the first process valve;
(c) a process chamber interposed between the first inner port and the second inner port, wherein the process chamber is in flow communication with the first process valve and with the second process valve, and wherein the process chamber has internal chamber surfaces; and
(d) one or more UV light sources configured to (i) expose ultraviolet light to at least 99% of chamber internal surface area defined by total surface area of the internal chamber surfaces and (ii) expose the ultraviolet light to at least 99% of valve internal surface area defined by total surface area of all internal, wettable valve surfaces within the first process valve and the second process valve.

The one or more UV light sources may be a single UV light source, or a plurality of UV light sources. The number of individual UV light sources can vary widely, such as 1, 2, 3, 4, 5, 6, 7, 8, 9, 10, 25, 50, 100, or more. For example, a first UV light source may be used to expose UV light to the process chamber; a second UV light source may be used to expose UV light to the first process valve; a third UV light source may be used to expose UV light to the second process valve. In this specification, when it is stated that one or more UV light sources are configured in a certain way, or that one or more UV light sources function in a certain way, this means that at least a single UV light source is so configured or functional. Multiple (but less than all) of the UV light sources may be so configured or functional, or all of the UV light sources may be so configured or functional.

While the UV light sources are preferably configured to expose ultraviolet light to at least 99% of the chamber internal surface area and the valve internal surface area, in certain embodiments there is less UV exposure. In various embodiments, one or more UV light sources are configured to expose ultraviolet light to at least 80%, 85%, 90%, 95%, 99%, 99.5%, 99.9%, or 100% of the chamber internal surface area. In various embodiments, one or more UV light sources (which may be the same or different from the UV light sources for the chamber internal surface area) are configured to expose ultraviolet light to at least 80%, 85%, 90%, 95%, 99%, 99.5%, 99.9%, or 100% of the valve internal surface area. In various embodiments, one or more UV light sources are configured to expose ultraviolet light to at least 80%, 85%, 90%, 95%, 99%, 99.5%, 99.9%, or 100% of the combined sum of the chamber internal surface area and the valve internal surface area. In certain embodiments, one or more UV light sources are configured to expose ultraviolet light to 100% of the chamber internal surface area and/or to 100% of the valve internal surface area.

In some embodiments, the first process valve is selected from the group consisting of a butterfly valve, a diaphragm valve, a ball valve, a globe valve, a gate valve, and a check valve.

In some embodiments, the second process valve is selected from the group consisting of a butterfly valve, a diaphragm valve, a ball valve, a globe valve, a gate valve, and a check valve.

The transfer of material between first and second process valves may be accomplished by the application of a driving force for transfer (e.g., pumping) or as a result of an arrangement such that the driving force already exists (e.g., a pressure differential or a difference in liquid head). The opening or closing of process valves may be accomplished manually by, for example, turning a lever, or via automatic actuation by, for example, electronic or pneumatic signals.

In some embodiments, the first process valve, the second process valve, and the process chamber are fabricated from materials that are substantially chemically inert to the ultraviolet light. By “substantially chemically inert”, it is meant that the ultraviolet light from the UV light source(s) does not cause the material to chemically degrade (e.g., depolymerize, or undergo crosslinking) during normal operations. Examples of materials that are substantially chemically inert to UV light include, but are not limited to, silicone, crosslinked polyethylene/polypropylene, polytetrafluoroethylene, fluoroelastomers, and polyurethanes.

In some embodiments, the process interconnection further comprises a third process valve having a third outer port and a third inner port, wherein the third process valve is in flow communication with the process chamber, the first process valve, and the second process valve.

Some embodiments relate to a double-block-and-bleed configuration. In this common configuration, the first process valve and the second process valve each function as “block” valves, and the third process valve functions as a bleed valve (also known as a drain valve). The bleed valve is located between the two block valves, in an isolation region. Two inline block valves—the first process valve and the second process valve—provide primary and secondary isolation barriers. With both valves closed, the bleed valve—the third process valve—is opened. A double-block-and-bleed system is an important safety and sterility configuration used in bioprocessing to ensure the complete and verifiable isolation of a bioreactor. The double-block-and-bleed system provides a redundant barrier to prevent cross contamination or accidental release of hazardous materials during maintenance, repair, or process changeovers.

The bleed valve may be used to ensure the isolation region is completely drained and sterilized, preventing microbial contamination. The bleed valve may be used to safely vent any residual pressure or fluid that may have been trapped in the isolation region. The bleed valve may also be used for leak detection. If either of the block valves is leaking, the pressure or fluid will escape through the open bleed valve, providing an immediate way to confirm that the isolation is secure before proceeding.

In some embodiments, the process interconnection further comprises a third process valve having a third outer port and a third inner port, as well as a fourth process valve having a fourth outer port and a fourth inner port, wherein the third process valve is in flow communication with the process chamber, wherein the fourth process valve is in flow communication with the process chamber, and wherein the third process valve is in flow communication with the fourth process valve. The process chamber may be interposed between the third inner port and the fourth inner port. Alternatively, or additionally, a second process chamber may be interposed between the third inner port and the fourth inner port.

As noted previously, the process chamber should be broadly construed herein. In some embodiments, no chemistry or biochemistry occurs within the process chamber. In other embodiments, chemistry or biochemistry occurs within the process chamber, in which case the process chamber may also be referred to as a reaction chamber. In some embodiments, the process chamber is an open section of pipe. In some embodiments, the process chamber contains one or more static mixing elements. In some embodiments, the process chamber contains one or more heat-exchange elements. In some embodiments, the process chamber is a tank, or is a region within a tank. In some embodiments, the process chamber is a bioreactor, or is a reaction chamber within a bioreactor.

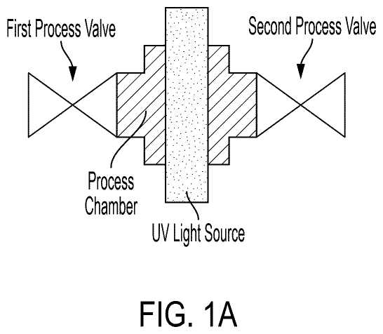

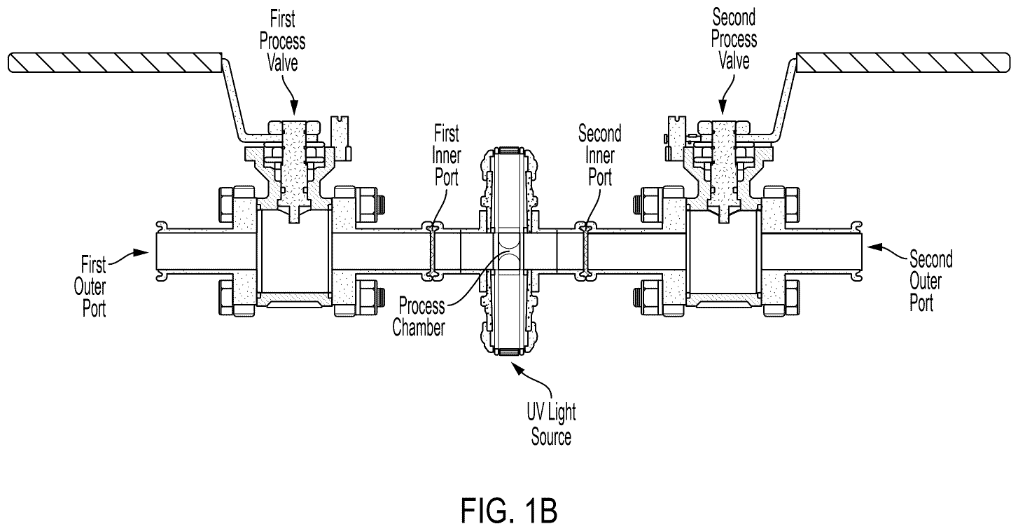

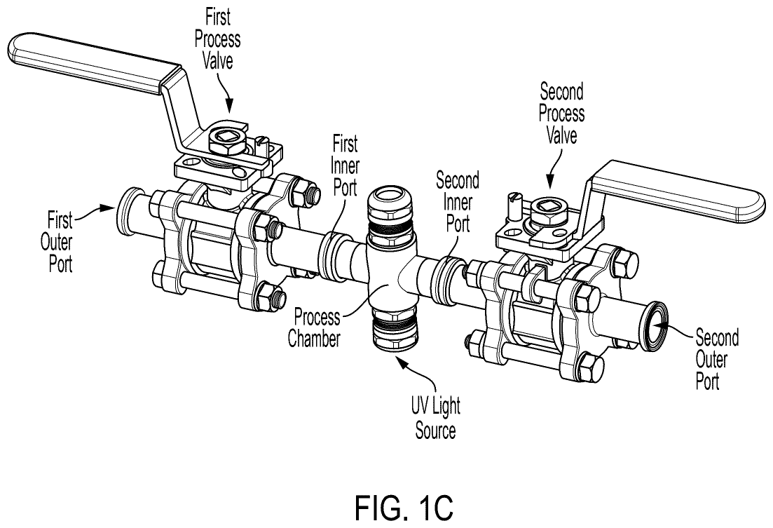

In certain embodiments (e.g., see FIGS. 1B and 1C), the process chamber is located within a tri-clamp cross configured to contain the UV light source in a direction perpendicular to a direction of flow from the first inner port to the second inner port.

In some embodiments, the first inner port is connected to the process chamber using a first sanitary connection, and the second inner port is connected to the process chamber using a second sanitary connection. The first sanitary connection and/or the second sanitary connection may be compliant with American Society of Mechanical Engineers (ASME) standards for sanitary fittings, for example.

In some embodiments, the first inner port is connected to the process chamber using a first reversible connection, and the second inner port is connected to the process chamber using a second reversible connection. Reversible (non-permanent) connections allow for a port or valve to be connected to the process chamber in a reversible and convenient manner to enable process flexibility, such as via the use of a clamp or flange-style connection.

In some embodiments, the first inner port is connected to the process chamber using a first reversible sanitary connection, and the second inner port is connected to the process chamber using a second reversible sanitary connection. The first reversible sanitary connection and/or the second reversible sanitary connection may be compliant with ASME standards for sanitary fittings, for example. In certain embodiments, the reversible sanitary connections utilize tri-clamp fittings.

In certain embodiments, the first process valve and the second process valve are each equipped with one or more valve seals. In some embodiments, a first seal is positioned between the first inner port and the process chamber, and a second seal is positioned between the second inner port and the process chamber. The valve seals may be substantially chemically inert to the ultraviolet light. The valve seal materials are not necessarily transparent or translucent to UV light.

Internal valve seal surfaces that are wettable may get contaminated. Valve seals include valve stem seals, for example. In some embodiments, one or more UV light sources are configured to expose the ultraviolet light to at least 90% of the valve-seal internal surface area defined by total surface area of all internal, wettable valve seal surfaces within the first process valve and the second process valve. In certain methods, the one or more UV light sources are configured to expose the ultraviolet light to 95%, 99%, 99.5%, 99.9%, or 100% of the valve-seal internal surface area.

In some embodiments, the wettable surfaces (including inner chamber surfaces, optionally the inner valve surfaces, and optionally the valve-seal surfaces) have a surface quality sufficient to be sterilizable by UV radiation. A surface treatment may be used to achieve a low surface roughness. Electropolishing, sanding, smoothing, or other types of surface treatment may be applied to the wettable surfaces intended for exposure to UV light. Essentially, smooth surfaces make it harder for a microorganism to hide from UV light.

In some embodiments, the first inner port is directly joined to the process chamber via welding, sintering, soldering, brazing, additive manufacturing, mechanical fasteners, or a combination thereof. Mechanical fasteners include bolts, rivets, screws, pins, etc. In some embodiments, a seal is also present along with a mechanical fastener at the first inner port.

In some embodiments, the second inner port is directly joined to the process chamber via welding, sintering, soldering, brazing, additive manufacturing, mechanical fasteners, or a combination thereof. In some embodiments, a seal is also present along with a mechanical fastener at the second inner port.

While connections using welding, sintering, soldering, brazing, additive manufacturing, and/or mechanical fasteners may be referred to as “permanent” connections, it will be understood that essentially any connection can be physically cut if desired.

In certain embodiments, the first inner port and second inner port are both directly joined to the process chamber via welding, sintering, soldering, brazing, additive manufacturing, mechanical fasteners, or a combination thereof. In these embodiments, a single integrated section of pipe may result; the first and second inner ports may no longer be observable from the outside of the ports—but may be observable using X-ray analysis or other imaging.

In some embodiments, the one or more UV light sources are positioned externally to the process chamber, the first process valve, and the second process valve. In these embodiments, the process chamber is preferably configured with chamber walls that are substantially transparent or translucent to the ultraviolet light, and the first process valve and the second process valve are preferably configured with valve walls that are substantially transparent or translucent to the ultraviolet light. Exemplary materials for the chamber and valve include, but are not limited to, quartz, perfluoroalkoxy alkanes, ethylene tetrafluoroethylene, ethylene chlorotrifluoroethylene, hexafluoropropylene-tetrafluoroethylene copolymers (also known as fluorinated ethylene propylene), and polyvinylidene fluoride.

In some embodiments, the first process valve and/or the second process valve are configured with valve walls that contain an internal UV-reflective coating disposed on a metal. A UV-reflective coating can assist UV sterilization by causing UV photons to bounce around and impact more surface area. Ordinary stainless steel reflects only about one-third of UV light. One example of a UV-reflective coating is a polytetrafluoroethylene coating, which can reflect >90% of UV light. Some embodiments utilize polytetrafluoroethylene-coated stainless steel for one or more process valves and/or for the process chamber.

In some embodiments, the one or more UV light sources are positioned internally within the process chamber. Optionally, the one or more UV light sources are located inside a sleeve that is positioned inside the process chamber, wherein the sleeve is substantially transparent or translucent to the ultraviolet light. In some embodiments, the sleeve is substantially chemically inert to the ultraviolet light. Example materials for the sleeve include, but are not limited to, quartz, perfluoroalkoxy alkanes, ethylene chlorotrifluoroethylene, ethylene tetrafluoroethylene, hexafluoropropylene-tetrafluoroethylene copolymers (also known as fluorinated ethylene propylene), and polyvinylidene fluoride.

In some embodiments, the process chamber is a vessel configured to contain a bioprocessing material. The process chamber may be configured with one or more additional sanitary ports, beyond any sanitary ports otherwise present in the system.

The process interconnection may be contained in a system configured to carry out a biological process. Alternatively, the process interconnection may be contained in a system configured to carry out a non-biological process. A “biological process” means a process that utilizes at least one biological reaction involving a living microorganism, to convert a substrate into a product. A “non-biological process” means a process that does not utilizes a biological reaction involving a living microorganism, to convert a substrate into a product. A non-biological process may employ chemical catalysts, for example. Even in a non-biological process, contamination by microorganisms can occur and can be prevented using the disclosed technology.

The process interconnection may be contained in a system configured to carry out material sterilization (e.g., media sterilization). In typical embodiments, the process interconnection is not part of a water UV sterilization system. In this specification, a “water UV sterilization system” is a system configured to disinfect water from a city water or well water source, such as for household purposes (e.g., drinking and cooking).

Other variations provide a system for bioprocessing, the system comprising:
(a) a bioreactor comprising a bioreactor chamber configured to carry out a reaction;
(b) a bioreactor port configured for introducing bioreaction media to the bioreactor chamber;
(c) a first process valve comprising a first outer port and a first inner port, wherein the first process valve is in flow communication with the bioreactor port;
(d) a process chamber interposed between the first inner port and the bioreactor port, wherein the process chamber is in flow communication with the first process valve and with the bioreactor port, and wherein the process chamber has internal chamber surfaces; and
(e) one or more UV light sources configured to (i) expose ultraviolet light to at least 99% of chamber internal surface area defined by total surface area of the internal chamber surfaces and (ii) expose the ultraviolet light to at least 99% of first-valve internal surface area defined by total surface area of all internal, wettable valve surfaces within the first process valve.

In various embodiments, one or more UV light sources are configured to expose ultraviolet light to at least 80%, 85%, 90%, 95%, 99%, 99.5%, 99.9%, or 100% of the chamber internal surface area. In various embodiments, one or more UV light sources (which may be the same or different from the UV light sources for the chamber internal surface area) are configured to expose ultraviolet light to at least 80%, 85%, 90%, 95%, 99%, 99.5%, 99.9%, or 100% of the first-valve internal surface area. In various embodiments, one or more UV light sources are configured to expose ultraviolet light to at least 80%, 85%, 90%, 95%, 99%, 99.5%, 99.9%, or 100% of the combined sum of the chamber internal surface area and the first-valve internal surface area. In certain embodiments, the one or more UV light sources are configured to expose ultraviolet light to 100% of the chamber internal surface area and/or to 100% of the first-valve internal surface area.

In some embodiments, the first process valve and the process chamber are fabricated from materials that are substantially chemically inert to the ultraviolet light. Exemplary materials for the first process valve and the process chamber include, but are not limited to, quartz, perfluoroalkoxy alkanes, ethylene tetrafluoroethylene, ethylene chlorotrifluoroethylene, hexafluoropropylene-tetrafluoroethylene copolymers (also known as fluorinated ethylene propylene), and polyvinylidene fluoride.

In some embodiments, the bioreactor port is a sanitary bioreactor port, such as one that is compliant with ASME standards for sanitary fittings.

In some embodiments, the system further comprises a second process valve interposed between the process chamber and the bioreactor port, wherein the second process valve comprises a second outer port and a second inner port, wherein the second process valve is in flow communication with the first process valve and with the bioreactor chamber, and wherein the one or more UV light sources are configured to expose the ultraviolet light to at least 99% of second-valve internal surface area defined by total surface area of all internal, wettable valve surfaces within the second process valve. In various embodiments, one or more UV light sources (which may be the same or different from the UV light sources for the chamber internal surface area or the first process valve) are configured to expose ultraviolet light to at least 80%, 85%, 90%, 95%, 99%, 99.5%, 99.9%, or 100% of the second-valve internal surface area.

In some embodiments, the system further comprises a third process valve having a third outer port and a third inner port, wherein the third process valve is in flow communication with the process chamber, the first process valve, and the second process valve. Additional valves may be present (e.g., a fourth valve, a fifth valve, a sixth valve, etc.). The additional process valves may also be configured to expose the ultraviolet light to at least 99% of additional-valve internal surface area defined by total surface area of all internal, wettable valve surfaces within the additional process valve(s).

In some embodiments, the bioreactor comprises a component configured for introducing a gas into the bioreactor chamber. The gas may be air, oxygen, nitrogen, argon, carbon dioxide, carbon monoxide, syngas, methane, hydrogen, ammonia, another gas, or a combination thereof.

In some embodiments, the bioreactor comprises one or more bioreactor UV light sources configured to expose additional ultraviolet light to surfaces within the bioreactor chamber. See U.S. Patent App. Pub. No. 2023/0313111 A1, published on Oct. 5, 2023, which is hereby incorporated by reference for its teachings of systems configured for exposing UV light onto internal surfaces within a bioreactor chamber.

In some embodiments, the one or more UV light sources are positioned externally to the process chamber and the first process valve, wherein the process chamber is configured with chamber walls that are substantially transparent or translucent to the ultraviolet light, and wherein the first process valve is configured with valve walls that are substantially transparent or translucent to the ultraviolet light.

In some embodiments, the one or more UV light sources are positioned internally within the process chamber (e.g., see FIG. 1A). A UV light source that is positioned internally with the process chamber may be contained with a sleeve or other protective shield that avoids direct contact between the surface of the UV bulk and the contents of the process chamber. In these embodiments, the sleeve or protective shield needs to be at least somewhat transparent or translucent to UV light at the selected wavelength.

Other variations provide a system for sterilizing bioprocessing media, the system comprising:
(a) a vessel configured for containing bioprocessing media;
(b) a vessel port configured for introducing the bioprocessing media to the vessel and/or for withdrawing the bioprocessing media from the vessel;
(c) a first process valve comprising a first outer port and a first inner port, wherein the first process valve is in flow communication with the vessel port;
(d) a process chamber in flow communication with the first process valve and the vessel port, wherein the process chamber has internal chamber surfaces and a process-chamber flow path through which the bioprocessing media must pass for the bioprocessing media to be introduced to and/or withdrawn from the vessel; and
(e) one or more UV light sources configured to expose ultraviolet light to at least 99% of the process-chamber flow path.

The “process-chamber flow path” is the three-dimensional volume in space in which the bioprocessing media is capable of being present within the process chamber, while the bioprocessing media continuously or semi-continuously flows into or out of the vessel. The process-chamber flow path includes open space as well as spaces immediately adjacent to internal chamber surfaces. In various embodiments, the one or more UV light sources are configured to expose ultraviolet light to 90%, 95%, 99%, 99.5%, 99.9%, 99.99%, 99.999%, 99.9999%, or 100% of the process-chamber flow path.

In some embodiments, one or more UV light sources are configured to also expose the ultraviolet light to chamber internal surface area and/or to valve internal surface area. However, for media sterilization, it is not necessary that the process chamber and valves are pre-sterilized by UV prior to flowing the media through the process chamber. For example, the media sterilizer may be steam-sterilized, such that the process chamber and valves (still without any media flow) are sterile prior to media sterilization. Then, the UV light source may be activated, and the media may be sterilized within the process-chamber flow path.

In some embodiments, one or more UV light sources are configured to expose the ultraviolet light to at least 99% of chamber internal surface area defined by total surface area of the internal chamber surfaces. In various embodiments, one or more UV light sources are configured to expose ultraviolet light to at least 80%, 85%, 90%, 95%, 99%, 99.5%, 99.9%, or 100% of the chamber internal surface area.

In some embodiments, one or more UV light sources are configured to expose the ultraviolet light to at least 99% of first-valve internal surface area defined by total surface area of all internal, wettable valve surfaces within the first process valve. In various embodiments, one or more UV light sources are configured to expose ultraviolet light to at least 80%, 85%, 90%, 95%, 99%, 99.5%, 99.9%, or 100% of the first-valve internal surface area.

In various embodiments, one or more UV light sources are configured to expose ultraviolet light to at least 80%, 85%, 90%, 95%, 99%, 99.5%, 99.9%, or 100% of the combined sum of the chamber internal surface area and the first-valve internal surface area.

In certain embodiments, the one or more UV light sources are configured to expose the ultraviolet light to at least 99% of chamber internal surface area defined by total surface area of the internal chamber surfaces, and to expose the ultraviolet light to at least 99% of first-valve internal surface area defined by total surface area of all internal, wettable valve surfaces within the first process valve. In certain embodiments, the one or more UV light sources are configured to expose ultraviolet light to 100% of the chamber internal surface area and/or to 100% of the first-valve internal surface area.

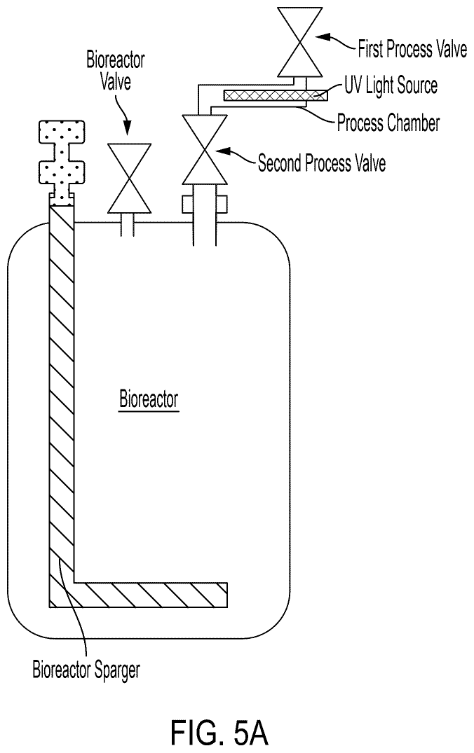

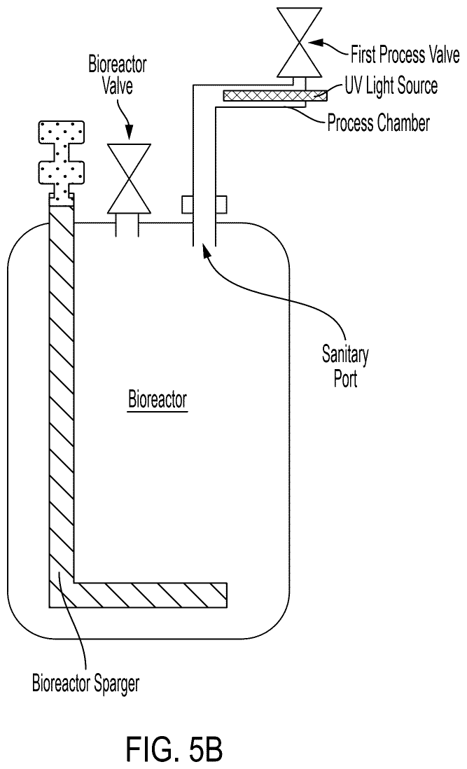

In some embodiments, the process chamber is interposed between the first inner port (on the first process valve) and the vessel port. An example of this configuration is shown in FIG. 5B, in which the vessel port is depicted as a sanitary port.

In some embodiments, the vessel port is a sanitary vessel port. A sanitary vessel port can be useful to create a cleanable and non-contaminating connection in media sterilization systems. The sanitary vessel port may be compliant with ASME standards for sanitary ports, for example.

In certain embodiments, the vessel port itself is exposed to one or more UV light sources. However, when a sanitary vessel port is utilized, the vessel port typically does not need to be exposed to UV light since the material entering the port has already been UV-sterilized (e.g., see FIG. 5B).

In some embodiments, the system further comprises a second process valve interposed between the process chamber and the vessel port, wherein the second process valve comprises a second outer port and a second inner port, wherein the second process valve is in flow communication with the first process valve and with the vessel, and wherein the one or more UV light sources are optionally configured to expose the ultraviolet light to at least 99% of second-valve internal surface area defined by total surface area of all internal, wettable valve surfaces within the second process valve.

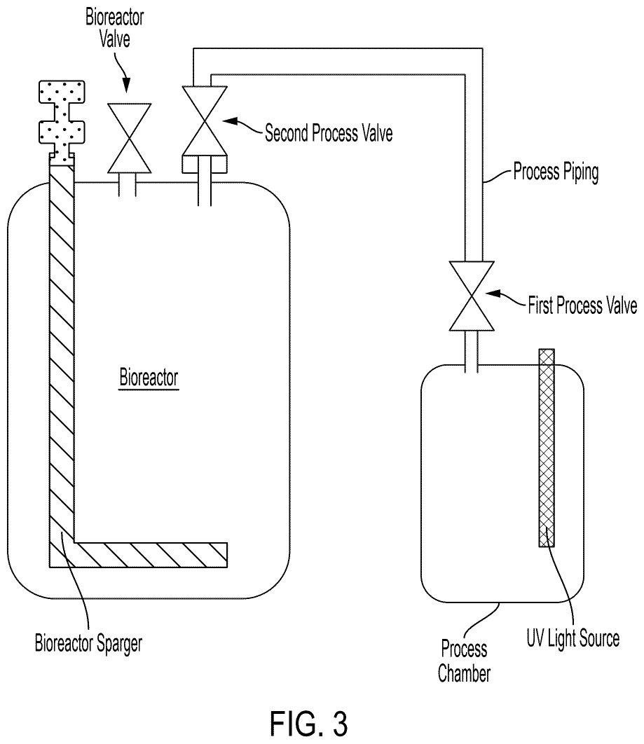

In some embodiments, the process chamber is in flow communication with the first outer port, and the process chamber is not interposed between the first inner port and the vessel port. An example of this configuration is shown in FIG. 3.

In some embodiments, the vessel is a bioreactor comprising a bioreactor chamber configured to carry out a reaction involving the bioprocessing media. The reaction may be aerobic fermentation, microaerobic fermentation, anaerobic fermentation, cell-free fermentation using enzymes, other enzymatic reactions, or a combination thereof, for example.

In some embodiments, the vessel is a tank configured to contain the bioprocessing media. In this specification, a “tank” should be broadly construed to mean a closed container of arbitrary geometry that stores the bioprocessing media for a period of time. The tank may or may not have means for agitation. In some system embodiments, the tank further comprises one or more tank UV light sources configured to expose ultraviolet light to surfaces within the tank.

In some embodiments, a filtration system is positioned in flow communication with the first process valve. The filtration system may be positioned between the first inner port of the first process valve and the process chamber, such that material is filtered after flowing through the first process valve. Alternatively, or additionally, the filtration system may be positioned on the upstream side of the first outer port of the first process valve, such that material is filtered prior to entering the first process valve. A filtration system may be useful to remove large particles, or clumps of particles, that may not be effectively UV-sterilized in the process chamber. The filtration system may be a polymeric filter, a ceramic filter, a membrane unit, a molecular-sieve unit, or a combination thereof, for example. The filtration system may have an average pore size from about 0.01 microns to about 20 microns, such as from about 0.2 microns to about 5 microns. An exemplary filtration system is a sterile air filter with a pore size of about 0.2 microns. Another exemplary filtration system is a media filter with a pore size of about 5 microns.

In some embodiments, an in-line sonicator is positioned in flow communication with the first process valve. The in-line sonicator may be positioned between the first inner port of the first process valve and the process chamber, such that material is sonicated after flowing through the first process valve. Alternatively, or additionally, the in-line sonicator may be positioned on the upstream side of the first outer port of the first process valve, such that material is sonicated prior to entering the first process valve. A sonicator uses acoustic energy to break up particle clumps by creating and collapsing microscopic bubbles, causing acoustic cavitation. The intense shockwaves and shear forces generated by the collapsing bubbles mechanically break apart agglomerates. A sonicator may be useful to remove large particles, or clumps of particles, that may not be effectively UV-sterilized in the process chamber.

In certain embodiments, both a filtration system and an in-line sonicator are utilized. For example, an in-line sonicator and then a filter may be positioned between the first inner port of the first process valve and the process chamber, such that material is sonicated and then filtered after flowing through the first process valve. Alternatively, or additionally, an in-line sonicator and then a filter may be positioned on the upstream side of the first outer port of the first process valve, such that material is sonicated and then filtered prior to entering the first process valve. It is also possible to place an in-line sonicator on the upstream side of the first outer port, and a filter between the first inner port and the process chamber.

In some embodiments, one or more UV light sources are positioned externally to the process chamber and the first process valve. In these embodiments, the process chamber is preferably configured with chamber walls that are substantially transparent or translucent to the ultraviolet light, and the first process valve is preferably configured with valve walls that are substantially transparent or translucent to the ultraviolet light.

In some embodiments, one or more UV light sources are positioned internally within the process chamber (e.g., see FIG. 1B). A UV light source that is positioned internally with the process chamber may be contained with a sleeve or other protective shield that avoids direct contact between the surface of the UV bulk and the contents of the process chamber. In these embodiments, the sleeve or protective shield needs to be at least somewhat transparent or translucent to UV light at the selected wavelength.

Some exemplary embodiments will now be further described in reference to the accompanying drawings, which should not be construed to limit the invention.

FIG. 1A (side view) depicts an exemplary process interconnection comprising a first process valve, a second process valve, a process chamber interposed between the first process valve and the second process valve, and a UV light source configured to expose ultraviolet light to internal surfaces within the process chamber and preferably configured to expose ultraviolet light to internal surfaces within the first and second process valves.

FIG. 1B (side view) depicts an exemplary process interconnection comprising a first process valve with a first outer port and a first inner port; a second process valve with a second outer port and a second inner port, wherein the second process valve is in flow communication with the first process valve; a process chamber interposed between the first inner port and the second inner port, wherein the process chamber has internal chamber surfaces; and a UV light source configured to expose ultraviolet light to the internal chamber surfaces.

FIG. 1C (isometric 3D view) depicts an exemplary process interconnection comprising a first process valve with a first outer port and a first inner port; a second process valve with a second outer port and a second inner port, wherein the second process valve is in flow communication with the first process valve; a process chamber interposed between the first inner port and the second inner port, wherein the process chamber has internal chamber surfaces; and a UV light source configured to expose ultraviolet light to the internal chamber surfaces.

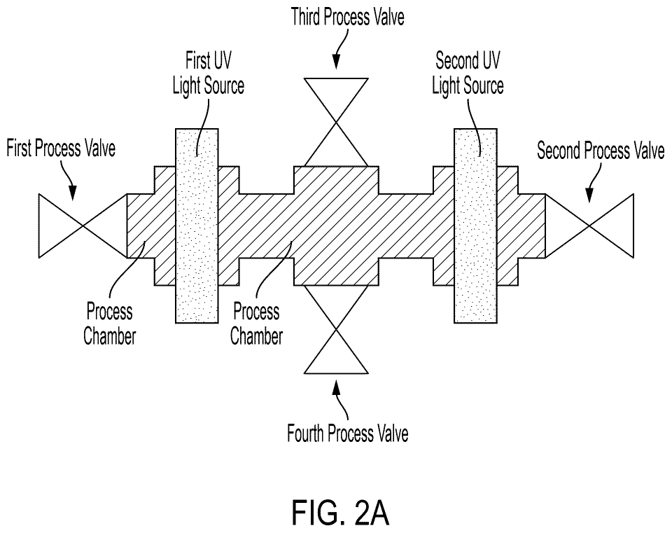

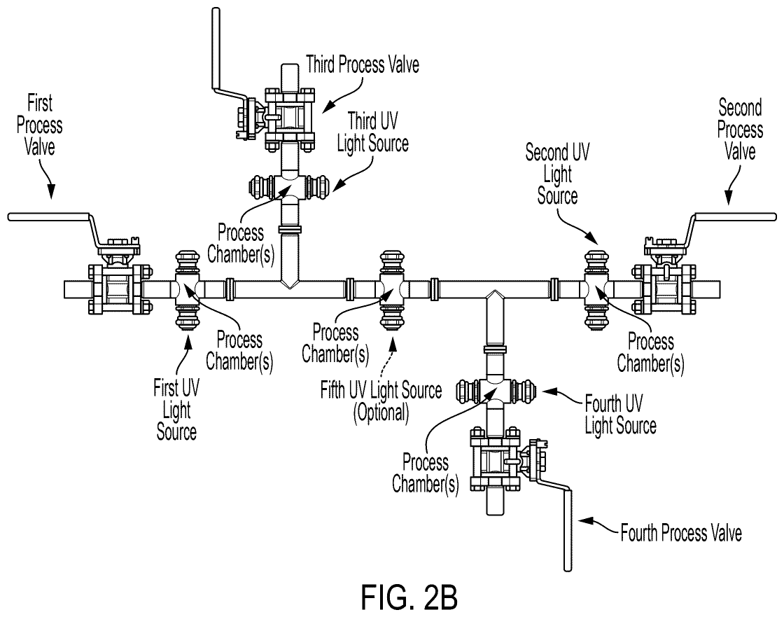

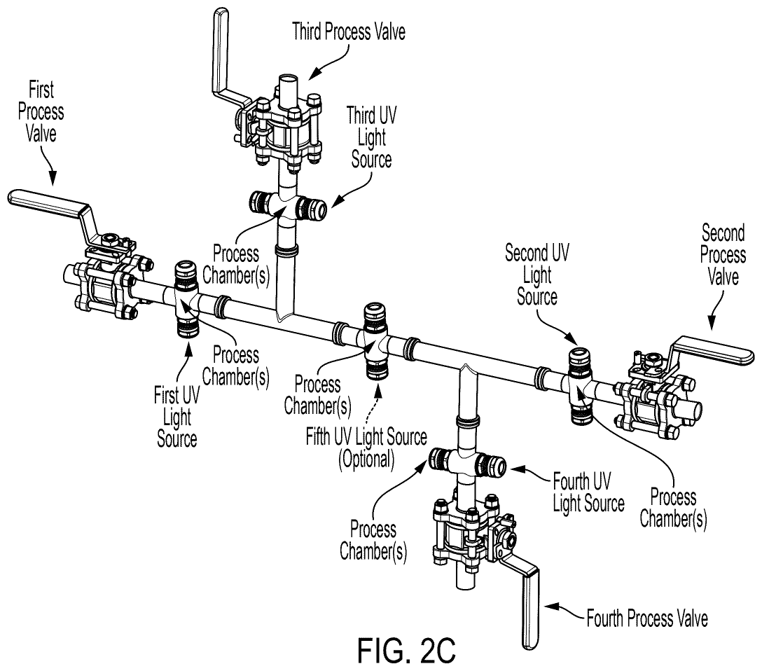

FIG. 2A (side view) depicts an exemplary process interconnection comprising a first process valve, a second process valve, a third process valve, a fourth process valve, a process chamber interposed between the first process valve and the second process valve and interposed between the third process valve and the fourth process valve, a first UV light source configured to expose ultraviolet light to internal surfaces within the process chamber and within the first process valve, and a second UV light source configured to expose ultraviolet light to internal surfaces within the process chamber and within the second process valve.

FIG. 2B (side view) depicts an exemplary process interconnection comprising a first process valve, a second process valve, a third process valve, a fourth process valve, multiple process chambers, a first UV light source configured to expose ultraviolet light to internal surfaces within a first process chamber and within the first process valve, a second UV light source configured to expose ultraviolet light to internal surfaces within a second process chamber and within the second process valve, a third UV light source configured to expose ultraviolet light to internal surfaces within a third process chamber and within the third process valve, a fourth UV light source configured to expose ultraviolet light to internal surfaces within a fourth process chamber and within the fourth process valve, and optionally a fifth UV light source configured to expose ultraviolet light to internal surfaces within a fifth process chamber.

FIG. 2C (isometric 3D view) depicts an exemplary process interconnection comprising a first process valve, a second process valve, a third process valve, a fourth process valve, multiple process chambers, a first UV light source configured to expose ultraviolet light to internal surfaces within a first process chamber and within the first process valve, a second UV light source configured to expose ultraviolet light to internal surfaces within a second process chamber and within the second process valve, a third UV light source configured to expose ultraviolet light to internal surfaces within a third process chamber and within the third process valve, a fourth UV light source configured to expose ultraviolet light to internal surfaces within a fourth process chamber and within the fourth process valve, and optionally a fifth UV light source configured to expose ultraviolet light to internal surfaces within a fifth process chamber.

FIG. 3 (side view) depicts an exemplary process interconnection comprising a first process valve, a second process valve, a process chamber configured with a UV light source to expose ultraviolet light to internal surfaces within the process chamber, a bioreactor equipped with a bioreactor sparger and a bioreactor valve, and process piping connecting the first process valve to a second process valve in fluid communication with the bioreactor.

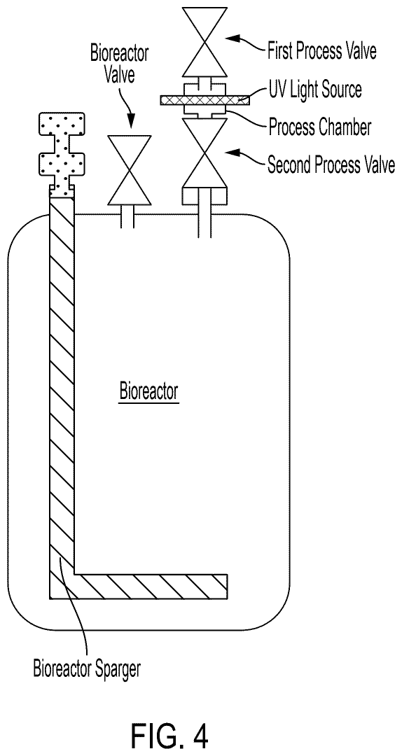

FIG. 4 (side view) depicts an exemplary process interconnection comprising a first process valve, a second process valve, a process chamber interposed between the first process valve and the second process valve, and a UV light source configured to expose ultraviolet light to internal surfaces within the process chamber, wherein the second process valve is directly connected to a bioreactor equipped with a bioreactor sparger and a bioreactor valve.

FIG. 5A (side view) depicts an exemplary system comprising a bioreactor with a bioreactor sparger and a bioreactor valve, a first process valve, a second process valve, a process chamber interposed between the first process valve and the second process valve, and a UV light source configured to expose ultraviolet light to internal surfaces within the process chamber. The second process valve is directly connected to the bioreactor.

FIG. 5B (side view) depicts an exemplary system comprising a bioreactor with a bioreactor sparger and a bioreactor valve, a first process valve, a sanitary port (no valve), a process chamber interposed between the first process valve and the sanitary port, and a UV light source configured to expose ultraviolet light to internal surfaces within the process chamber. The sanitary port is directly connected to the bioreactor.

Some variations provides methods of using ultraviolet light for sterilization of process interconnections, process chambers, or bioprocessing media. These methods may be conducted continuously, semi-continuously, or in batch.

There are many methods that may utilize the disclosed systems. Methods may involve sterile material transfer into a process unit, sterile material transfer out of a process unit, sterilization of a material while being conveyed into a process unit, sterilization of a material following by storage in a tank, the creation of a sterile boundary within a process unit, the creation of a sterile boundary around a process unit, and combinations of the foregoing.

In this disclosure, a “material” shall be broadly construed and may be a vapor/gas, a liquid, a slurry, a paste, or a solid. An example of a vapor is air. An example of a liquid is a liquid media. An example of a slurry is a broth containing an insoluble product. An example of a paste is a thick broth with cells and containing an insoluble product. An example of a solid is sucrose powder being conveyed pneumatically into a bioreactor.

Unless otherwise stated, the order of method steps may be varied, to accomplish the intended objective. When two valves are to be opened, generally they can be opened in either sequence or simultaneously, unless stated otherwise. In certain embodiments, the order of valve opening or closing is important, as described below.

In some variations, a method of sterile material transfer comprises:
(i) providing a process interconnection comprising: (a) a first process valve comprising a first outer port and a first inner port; (b) a second process valve comprising a second outer port and a second inner port, wherein the second process valve is in flow communication with the first process valve; (c) a process chamber interposed between the first inner port and the second inner port, wherein the process chamber is in flow communication with the first process valve and with the second process valve, and wherein the process chamber has internal chamber surfaces; and (d) one or more UV light sources configured to expose ultraviolet light to the internal chamber surfaces and to all internal, wettable valve surfaces within the first process valve and the second process valve;
(ii) providing a first sterile container that contains a sterile material to be transferred and a second sterile container that receives the sterile material, wherein the first sterile container is connected to the first outer port, and wherein the second sterile container is connected to the second outer port;
(iii) ensuring the first process valve and the second process valve are each in a closed position;
(iv) activating the one or more UV light sources to deliver the ultraviolet light to at least 99% of chamber internal surface area defined by total surface area of the internal chamber surfaces and to at least 99% of valve internal surface area defined by total surface area of all internal, wettable valve surfaces within the first process valve and the second process valve, using a UV dose effective to substantially deactivate adventitious microorganisms from the internal chamber surfaces and from the internal, wettable valve surfaces within the first process valve and the second process valve;
(v) opening the first process valve and the second process valve;
(vi) transferring the sterile material from the first sterile container to the second sterile container, through the process chamber; and
(vii) closing the first process valve and the second process valve.

In various embodiments, the one or more UV light sources expose the ultraviolet light to at least 80%, 85%, 90%, 95%, 99%, 99.5%, 99.9%, or 99.99% of the chamber internal surface area. In certain method embodiments, the one or more UV light sources expose the ultraviolet light to 100% of the chamber internal surface area.

In various embodiments, the one or more UV light sources expose the ultraviolet light to at least 80%, 85%, 90%, 95%, 99%, 99.5%, 99.9%, or 99.99% of the valve internal surface area. In certain method embodiments, the one or more UV light sources expose the ultraviolet light to 100% of the valve internal surface area.

In various embodiments, the one or more UV light sources expose the ultraviolet light to at least 80%, 85%, 90%, 95%, 99%, 99.5%, 99.9%, or 99.99% of the combined total of the chamber internal surface area and the valve internal surface area. In certain method embodiments, the one or more UV light sources expose the ultraviolet light to 100% of the chamber internal surface area and 100% of the valve internal surface area.

In some method embodiments, the UV dose is at least 100 mJ/cm², calculated as sterilization energy per unit total area in the process chamber. In certain methods, the UV dose is at least 200 mJ/cm² or at least 300 mJ/cm². Other potential UV doses are discussed later in this specification.

In any methods that also expose UV light to surfaces within the first and/or second process valves (in addition to the process chamber), the units of the UV dose are sterilization energy per unit total area in the process chamber plus total area in the UV-exposed process valves. The calculations use the wettable area, which means the area that can be wetted by the material being processed, and which area is actually wetted during process operation.

In some methods, step (iv) utilizes a total UV power capacity from about 0.1 W/m³ to about 10 W/m³. Other potential total UV power capacities are discussed later in this specification.

In some methods, step (iv) utilizes a UV sterilization time from about 10 seconds to about 2 hours. Other potential UV sterilization times are discussed later in this specification.

In some methods, the one or more UV light sources each have a UV wavelength selected from about 100 nm to about 400 nm. Specific UV wavelengths that may be employed are discussed later in this specification.

In some methods, step (iv) utilizes a total UV power output from about 0.1 mW/cm² to about 100 mW/cm². Other potential total UV power outputs are discussed later in this specification.

In some method embodiments, the sterile material is selected from the group consisting of a sugar, a salt, a vitamin, a mineral, a pH buffer, an antifoam, an alcohol, an acid, a base, water, air, carbon dioxide, carbon monoxide, syngas, methane, hydrogen, nitrogen, argon, ammonia, a solution of any one of the foregoing, and combinations thereof.

In some method embodiments, the first sterile container is a sterile holding tank, the second sterile container is a reactor, and the method transfers sterile media from the sterile holding tank into the reactor.

In some methods, the first sterile container contains a biological material to be sampled, the second sterile container is a sampling container configured to receive a biological material sample, and the method transfers the biological material sample from the first sterile container to the second sterile container.

In certain embodiments, the second container (connected to the second outer port) is not necessarily sterile.

In some methods, the first sterile container is a sterile reactor, the second container is an exhaust container, and the method exhausts one or more vapors from the sterile reactor to the exhaust container. The exhaust container may be a closed container or the environment.

In some methods, the first sterile container is a holding tank, the second container is an exhaust container, and the method exhausts one or more vapors from the holding tank to the exhaust container. The exhaust container may be a closed container or the environment.

In some methods, the first sterile container is configured to prevent backflow of adventitious microorganisms from the second container into the first sterile container by maintaining a positive pressure difference between the first sterile container and the second container. A backflow may occur due to a process upset, for example. The positive pressure difference may be maintained using a sterile gas injected into, and withdrawn from, the first sterile container. The sterile gas may be selected from the group consisting of sterile air, sterile nitrogen, sterile argon, sterile carbon dioxide, sterile hydrogen, sterile methane, sterile ammonia, and combinations thereof, for example.

During steps (v) and (vi), the UV light source may remain activated, if desired.

Ordinarily, it is not necessary to keep the UV light source on during steps (v) or (vi) since the material (coming from the first sterile container) is already sterile and the UV light has already sterilized the exposed surface (chamber surface area and valve internal surface area). However, keeping the UV light source on during step (v), step (vi), or both of these steps is not precluded, and may be performed as an additional process assurance, for example.

Preferably, step (iv) is effective to reach at least a 6-log reduction in the adventitious microorganisms. More preferably, step (iv) is effective to reach at least a 8-log reduction in the adventitious microorganisms. Most preferably, step (iv) is effective to reach at least a 10-log reduction in the adventitious microorganisms. An “adventitious microorganism” is any microorganism (e.g., yeast, bacteria, fungi, algae, or mold) that is not the desired microorganism (biocatalyst) for catalyzing the intended reaction. Adventitious microorganisms may also be referred to as contaminant microorganisms. Adventitious microorganisms can compete with the biocatalyst for resources, introduce undesirable properties, or generate side products within the bioreactor environment.

A culture that contains only one species, variety, or strain of microorganism within a cultivation medium is termed axenic. An “axenic culture” is therefore a pure culture of a single microorganism that is the desired microorganism for the intended process, without any contaminating microorganisms. An axenic culture differs from a sterile medium because in a sterile medium, even the desired microorganism has been deactivated or is not yet present. When processing an axenic culture for a bioreaction, it is usually not desired to deactivate the desired microorganisms, at least until following the intended bioreaction.

Some variations provide a method of axenic culture transfer, the method comprising:
(i) providing a process interconnection comprising: (a) a first process valve comprising a first outer port and a first inner port; (b) a second process valve comprising a second outer port and a second inner port, wherein the second process valve is in flow communication with the first process valve; (c) a process chamber interposed between the first inner port and the second inner port, wherein the process chamber is in flow communication with the first process valve and with the second process valve, and wherein the process chamber has internal chamber surfaces; and (d) one or more UV light sources configured to expose ultraviolet light to the internal chamber surfaces and to all internal, wettable valve surfaces within the first process valve and the second process valve;
(ii) providing a first container that contains an axenic culture to be transferred and a second container that receives the axenic culture, wherein the first container is connected to the first outer port, wherein the second container is connected to the second outer port, and wherein the second container either is sterile prior to receiving the axenic culture, or contains an initial quantity of the axenic culture;
(iii) ensuring the first process valve and the second process valve are each in a closed position;
(iv) activating the one or more UV light sources to deliver the ultraviolet light to at least 99% of chamber internal surface area defined by total surface area of the internal chamber surfaces and to at least 99% of valve internal surface area defined by total surface area of all internal, wettable valve surfaces within the first process valve and the second process valve, using a UV dose effective to substantially deactivate adventitious microorganisms from the internal chamber surfaces and from the internal, wettable valve surfaces within the first process valve and the second process valve;
(v) opening the first process valve and the second process valve;
(vi) transferring the axenic culture from the first container to the second container, through the process chamber; and
(vii) closing the first process valve and the second process valve.

In some embodiments of axenic culture transfer, the second container is sterile prior to receiving the axenic culture. In other embodiments, the second container is not sterile prior to receiving the axenic culture, but it is axenic: the second container contains an initial quantity of the axenic culture and no other microorganisms (or a very low concentration thereof).

In some embodiments of axenic culture transfer, the first container is a first axenic reactor, the second container is a second axenic reactor, and the method transfers the axenic culture from the first axenic reactor into the second axenic reactor.

In some embodiments of axenic culture transfer, the first container is an axenic reactor, and the second container is a reactor cooling loop. This cooling-loop method transfers reactor contents from the axenic reactor into the reactor cooling loop, and then transfers cooled reactor contents from the reactor cooling loop back to the axenic reactor.

The axenic culture may be selected from bacteria, yeasts, and filamentous fungi, for example. Specific examples include the yeasts Saccharomyces cerevisiae and Yarrowia lipolytica, the bacteria E. coli and Bacillus subtilis, and the filamentous fungi Candida famata.

In some methods for transferring axenic culture, step (iv) utilizes a total UV power capacity from about 0.1 W/m³ to about 10 W/m³. Other potential total UV power capacities are discussed later in this specification.

In some methods for transferring axenic culture, step (iv) utilizes a UV sterilization time from about 10 seconds to about 2 hours. Other potential UV sterilization times are discussed later in this specification.

In some methods for transferring axenic culture, the one or more UV light sources each have a UV wavelength selected from about 100 nm to about 400 nm. Specific UV wavelengths that may be employed are discussed later in this specification.

In some methods for transferring axenic culture, step (iv) utilizes a total UV power output from about 0.1 mW/cm² to about 100 mW/cm². Other potential total UV power outputs are discussed later in this specification.

Other variations provide a method of sterile bioprocessing, the method comprising:
(i) providing a bioreactor comprising a bioreactor chamber configured to carry out a reaction, wherein the bioreactor is configured with a bioreactor port for introducing bioreaction media to the bioreactor chamber;
(ii) providing a process interconnection comprising: (a) a first process valve comprising a first outer port and a first inner port, wherein the first process valve is in flow communication with the bioreactor port; (b) a process chamber interposed between the first inner port and the bioreactor port, wherein the process chamber is in flow communication with the first process valve and with the bioreactor port, and wherein the process chamber has internal chamber surfaces; and (c) one or more UV light sources configured to expose ultraviolet light to the internal chamber surfaces and to all internal, wettable valve surfaces within the first process valve;
(iii) operating the bioreactor containing the bioreaction media to carry out the reaction, to convert one or more substrates into one or more products; and
(iv) before, during, and/or after step (iii), activating the one or more UV light sources to expose the ultraviolet light to at least 99% of chamber internal surface area defined by total surface area of the internal chamber surfaces and at least 99% of first-valve internal surface area defined by total surface area of all internal, wettable valve surfaces within the first process valve, using a UV dose effective to substantially deactivate adventitious microorganisms, contained within the bioreaction media, from the internal chamber surfaces and from the first-valve internal surface area.

In various embodiments, one or more UV light sources are configured to expose ultraviolet light to at least 80%, 85%, 90%, 95%, 99%, 99.5%, 99.9%, or 99.99% of the chamber internal surface area. In various embodiments, one or more UV light sources are configured to expose ultraviolet light to at least 80%, 85%, 90%, 95%, 99%, 99.5%, 99.9%, or 99.99% of the first-valve surface area. In certain method embodiments, one or more UV light sources are configured to expose ultraviolet light to 100% of the chamber internal surface area and/or to 100% of the first-valve surface area.

In some method embodiments, the bioreactor port is a sanitary bioreactor port. A sanitary bioreactor port can be useful to create a cleanable and non-contaminating connection in bioreactors. The sanitary bioreactor port may be compliant with ASME standards for sanitary ports, for example.

In some method embodiments, the process interconnection further comprises a second process valve interposed between the process chamber and the bioreactor port; wherein the second process valve comprises a second outer port and a second inner port; wherein the second process valve is in flow communication with the first process valve and with the bioreactor chamber; wherein during step (iv), the one or more UV light sources expose the ultraviolet light to at least 99% of second-valve internal surface area defined by total surface area of all internal, wettable valve surfaces within the second process valve; and wherein the UV dose is effective to substantially deactivate adventitious microorganisms from the second-valve internal surface area.

In some method embodiments, the process interconnection further comprises a third process valve having a third outer port and a third inner port, wherein the third process valve is in flow communication with the process chamber, the first process valve, and the second process valve. An exemplary method utilizes a double-block-and-bleed configuration as described above.

In some method embodiments, during step (iv), the first process valve is closed. In other embodiments, during step (iv), the first process valve is open. The first process valve may be closed during step (iv), for example, when it is desired to sterilize the process chamber prior to bioreaction media flowing through it and entering the bioreactor. Another example of the first process valve being closed during step (iv) is when a quantity of bioreaction media has entered the process chamber and is to be batch-sterilized with UV prior to the bioreaction media being introduced to the bioreactor. The first process valve may be open during step (iv), for example, when it is desired to continuously or semi-continuously sterilize bioreaction media before it enters the bioreactor. Another example of the first process valve being open during step (iv) is when the bioreaction media is flowing out of the bioreactor and it is desired to sterilize the bioreaction media for safety or intellectual-property reasons.

In some method embodiments, the method further comprises pre-filtering the bioreaction media prior to exposing the bioreaction media to the ultraviolet light. The filtration system may be positioned between the first inner port of the first process valve and the process chamber, such that material is filtered after flowing through the first process valve. Alternatively, or additionally, the filtration system may be positioned on the upstream side of the first outer port of the first process valve, such that material is filtered prior to entering the first process valve.

In some method embodiments, the method further comprises sonicating the bioreaction media prior to exposing the bioreaction media to the ultraviolet light. The in-line sonicator may be positioned between the first inner port of the first process valve and the process chamber, such that material is sonicated after flowing through the first process valve. Alternatively, or additionally, the in-line sonicator may be positioned on the upstream side of the first outer port of the first process valve, such that material is sonicated prior to entering the first process valve.

In certain method embodiments, the method further comprises both sonicating and pre-filtering the bioreaction media prior to exposing the bioreaction media to the ultraviolet light.

In some methods of sterile bioprocessing, step (iv) utilizes a total UV power capacity from about 0.1 W/m³ to about 10 W/m³. Other potential total UV power capacities are discussed later in this specification.

In some methods of sterile bioprocessing, step (iv) utilizes a UV sterilization time from about 10 seconds to about 2 hours. Other potential UV sterilization times are discussed later in this specification.

In some methods of sterile bioprocessing, the one or more UV light sources each have a UV wavelength selected from about 100 nm to about 400 nm. Specific UV wavelengths that may be employed are discussed later in this specification.

In some methods of sterile bioprocessing, step (iv) utilizes a total UV power output from about 0.1 mW/cm² to about 100 mW/cm². Other potential total UV power outputs are discussed later in this specification.

In preferred method embodiments, step (iv) is effective to reach a 6-log reduction in the adventitious microorganisms within the process chamber and the first process valve. More preferably, step (iv) is effective to reach at least a 8-log reduction in the adventitious microorganisms. Most preferably, step (iv) is effective to reach at least a 10-log reduction in the adventitious microorganisms.

Still other variations provide a method of sterilizing a bioprocessing media, the method comprising:
(i) providing a vessel configured for containing bioprocessing media, wherein the vessel is configured with a vessel port for introducing the bioprocessing media to the vessel and/or for withdrawing the bioprocessing media from the vessel;
(ii) providing a process interconnection comprising: (a) a first process valve comprising a first outer port and a first inner port, wherein the first process valve is in flow communication with the vessel port; (b) a process chamber in flow communication with the first process valve and the vessel port, wherein the process chamber has internal chamber surfaces and a process-chamber flow path through which the bioprocessing media must pass for the bioprocessing media to be introduced to and/or withdrawn from the vessel; and (c) one or more UV light sources configured to expose ultraviolet light to the internal chamber surfaces;
(iii) adding the bioprocessing media to the vessel, from a media source vessel; or withdrawing the bioprocessing media from the vessel, to a media receiving vessel; and
(iv) during step (iii), activating the one or more UV light sources to expose the ultraviolet light to at least 99% of the process-chamber flow path, using a UV dose effective to substantially deactivate adventitious microorganisms contained within the bioprocessing media, thereby generating a sterilized bioprocessing media,
wherein step (iv) is effective to reach a 6-log reduction in the adventitious microorganisms within the process chamber.

In various embodiments, the one or more UV light sources are configured to expose ultraviolet light to 90%, 95%, 99%, 99.5%, 99.9%, 99.99%, 99.999%, 99.9999%, or 100% of the process-chamber flow path.

In some methods, one or more UV light sources are configured to also expose the ultraviolet light to chamber internal surface area and/or to valve internal surface area. However, for media sterilization, it is not necessary that the process chamber and valves are pre-sterilized by UV prior to flowing the bioprocessing media through the process chamber. For example, the media sterilizer may be steam-sterilized, such that the process chamber and valves (still without any media flow) are sterile prior to media sterilization. Then, the UV light source may be activated, and the bioprocessing media may be sterilized within the process-chamber flow path.

In various method embodiments, the one or more UV light sources are configured to expose the ultraviolet light to at least 90%, 95%, 99%, 99.5%, 99.9%, or 100% of chamber internal surface area defined by total surface area of the internal chamber surfaces.

In various method embodiments, the one or more UV light sources are configured to expose the ultraviolet light to at least 90%, 95%, 99%, 99.5%, 99.9%, or 100% of first-valve internal surface area defined by total surface area of all internal, wettable valve surfaces within the first process valve.

In various method embodiments, the one or more UV light sources are configured to expose the ultraviolet light to at least 90%, 95%, 99%, 99.5%, 99.9%, or 100% of the combined total of the chamber internal surface area and the first-valve internal surface area. In certain method embodiments, the one or more UV light sources are configured to expose the ultraviolet light to at least 99% of chamber internal surface area defined by total surface area of the internal chamber surfaces as well as at least 99% of first-valve internal surface area defined by total surface area of all internal, wettable valve surfaces within the first process valve.

In some method embodiments, the vessel port is a sanitary vessel port. The sanitary vessel port may be compliant with ASME standards for sanitary ports or fittings, for example.

In some method embodiments, the process interconnection further comprises a second process valve interposed between the process chamber and the vessel port; wherein the second process valve comprises a second outer port and a second inner port; and wherein the second process valve is in flow communication with the first process valve and with the vessel. In some method embodiments in which there is a second process valve, during step (iv), the one or more UV light sources expose the ultraviolet light to at least 99% of second-valve internal surface area defined by total surface area of all internal, wettable valve surfaces within the second process valve; and wherein the UV dose is effective to substantially deactivate adventitious microorganisms from the second-valve internal surface area. In various embodiments, the one or more UV light sources expose the ultraviolet light to at least 90%, 95%, 99%, 99.5%, 99.%, or 100% of second-valve internal surface area.

In some method embodiments, the bioprocessing media is selected from the group consisting of a sugar, a salt, a vitamin, a mineral, a pH buffer, an antifoam, an alcohol, an acid, a base, water, air, carbon dioxide, carbon monoxide, syngas, methane, hydrogen, nitrogen, argon, ammonia, a solution of any one of the foregoing, and combinations thereof.

In embodiments intended for media sterilization, the material in the first container is usually known or suspected to be non-sterile. However, the material in the first container may be a potentially non-sterile material, or a material whose sterility is unknown. The UV sterilization may be conducted as a precaution, for example.

In some sterilization methods, the one or more UV light sources each have a UV wavelength selected from about 100 nm to about 400 nm, for example.

In some sterilization methods, the first UV dose is at least 100 mJ/cm² (sterilization energy per unit total area in the process chamber). In certain methods, the first UV dose is at least 200 mJ/cm² or at least 300 mJ/cm².

In some sterilization methods, step (iv) utilizes a total UV power capacity from about 0.1 W/m³ to about 10 W/m³.

In some sterilization methods, step (iv) utilizes a UV sterilization time from about 10 seconds to about 2 hours.

In some sterilization methods, step (iv) utilizes a total UV power output from about 0.1 mW/cm² to about 100 mW/cm².

In some sterilization methods, step (iv) is effective to reach a 8-log reduction in the adventitious microorganisms within the process chamber. In certain methods, step (iv) is effective to reach a 10-log reduction in the adventitious microorganisms within the process chamber.

In some sterilization methods, the vessel is a bioreactor comprising a bioreactor chamber configured to carry out a reaction involving the sterilized bioprocessing media, and the method transfers sterilized bioprocessing media into the bioreactor through the vessel port.

In some sterilization methods, the vessel is a bioreactor comprising a bioreactor chamber configured to carry out a reaction involving the sterilized bioprocessing media, and the method transfers sterilized bioprocessing media out of the bioreactor through the vessel port. Sterilizing bioprocessing media being transferred out of the bioreactor may be performed for environmental health and safety reasons, for intellectual-property reasons, such as to protect a trade secret regarding a microorganism used in a bioreactor, for example.

The bioreactor may include a component configured for introducing a gas into the bioreactor chamber. The gas may be air, oxygen, nitrogen, argon, carbon dioxide, carbon monoxide, syngas, methane, hydrogen, ammonia, another gas, or a combination thereof. The gas may be a bioreaction substrate, a bioreaction catalyst, a bioreaction promotor, a bioreaction carrier gas, a bioreaction inert gas, or a bioreaction recycle gas, for example.

In some sterilization embodiments, the bioreactor comprises one or more bioreactor UV light sources configured to expose additional ultraviolet light to surfaces within the bioreactor chamber. See U.S. Patent App. Pub. No. 2023/0313111 A1, published on Oct. 5, 2023, which is hereby incorporated by reference for its teachings of methods of exposing UV light to internal surfaces within a bioreactor chamber.

In some sterilization methods, step (iii) comprises adding the bioprocessing media to the vessel, from the media source vessel; and the vessel is a tank configured to contain the sterilized bioprocessing media.

In some sterilization methods, step (iii) comprises withdrawing the bioprocessing media from the vessel, into the media receiving vessel; and the vessel is a tank configured to contain the bioprocessing media prior to being exposed to the ultraviolet light.

In some sterilization methods, step (iii) comprises withdrawing the bioprocessing media from the vessel; and the media receiving vessel is the environment. In this context, the environment may be a laboratory room, hood, or vent; a production plant; the atmosphere; or a body of water, for example.

In some sterilization methods, the method further comprises pre-filtering the bioprocessing media prior to exposing the bioprocessing media to the ultraviolet light. A filtration system may be positioned in flow communication with the first process valve. The filtration system may be positioned between the first inner port of the first process valve and the process chamber, such that material is filtered after flowing through the first process valve. Alternatively, or additionally, the filtration system may be positioned on the upstream side of the first outer port of the first process valve, such that material is filtered prior to entering the first process valve. A filtration system may be useful to remove large particles, or clumps of particles, that may not be effectively UV-sterilized in the process chamber. The filtration system may be a polymeric filter, a ceramic filter, a membrane unit, a molecular-sieve unit, or a combination thereof, for example. The filtration system may have an average pore size from about 0.01 microns to about 20 microns, such as from about 0.2 microns to about 5 microns. An exemplary filtration system is a sterile air filter with a pore size of about 0.2 microns. Another exemplary filtration system is a media filter with a pore size of about 5 microns.

In some sterilization methods, the method further comprises sonicating the bioprocessing media prior to exposing the bioprocessing media to the ultraviolet light. An in-line sonicator may be positioned in flow communication with the first process valve. The in-line sonicator may be positioned between the first inner port of the first process valve and the process chamber, such that material is sonicated after flowing through the first process valve. Alternatively, or additionally, the in-line sonicator may be positioned on the upstream side of the first outer port of the first process valve, such that material is sonicated prior to entering the first process valve. A sonicator uses acoustic energy to break up particle clumps by creating and collapsing microscopic bubbles, causing acoustic cavitation. The intense shockwaves and shear forces generated by the collapsing bubbles mechanically break apart agglomerates. A sonicator may be useful to remove large particles, or clumps of particles, that may not be effectively UV-sterilized in the process chamber.

In certain sterilization methods, both a filtration system and an in-line sonicator are utilized, such as to provide two means of removing large particles or particle clumps. When both filtration and sonication are used, typically the sequence is sonication followed by filtration, but the optimal sequence depends on filtration pore size and sonication parameters.

The UV sterilization described in this specification is suitable for replacing steam sterilization, which has significant disadvantages as discussed in the Background. However, it should be understood that steam sterilization can still be used in some circumstances and/or in locations not involving the UV light sources. One example is the process piping in FIG. 3, in which the process piping that connects the second process valve to the bioreactor may be steam-sterilized, in some embodiments. In other embodiments, a second UV light source is configured to expose ultraviolet light to surface area within the process piping. In certain embodiments, no steam sterilization is used in any step of a method.

It should also be recognized that steam is useful for process requirements other than sterilization—namely, heating and cleaning. Steam heating may be employed to heat up a bioreactor and/or maintain it at a desired operating temperature, or to heat up process inputs. Generation of steam may be utilized to capture waste heat in the process.

Certain variations of the invention are premised on the use of UV sterilization of a material being transferred through a process chamber, after prior steam sterilization of that process chamber.

Methods employing UV sterilization may take place following a cleaning procedure that is adequate to remove bulk contaminants and produce cleaned surfaces in preparation for sterilization by UV radiation. “Cleaning” refers to the removal of bulk materials (e.g., dead cells, residual media, or reaction byproducts) that cover a surface to be UV-irradiated. Unless the bulk material happens to be transparent or translucent to ultraviolet light, the UV sterilization would be rendered ineffective, or at least less effective, without cleaning of the bulk material. Cleaning may employ chemical cleaning agents, including (but not limited to) steam, hot water, a solvent, an acid, a base, an alkaline detergent, sodium hydroxide, sodium percarbonate, an acidic detergent, phosphoric acid, peracetic acid, isopropanol, ethanol, sodium hypochlorite, hydrogen peroxide, ethylene oxide, chlorine dioxide, ozone, formaldehyde, glutaraldehyde, or a combination thereof. Steam cleaning may be performed prior to UV sterilization. Cleaning may employ physical agents or forces, including (but not limited to) gravity, vibration, sonication, centrifugal forces, a mechanical scraper, or a combination thereof. Cleaning may use a combination of chemical and physical agents.

In some embodiments employing at least four process valves, at least two process valves are reserved for process lines responsible for the delivery and removal of cleaning solutions during a cleaning procedure that is distinct from UV sterilization.

In certain embodiments, the material to be transferred or sterilized is of sufficient quality such that no cleaning procedure is required. In certain embodiments, cleaning is not required before every transfer of material but rather is intermittently performed.

Various options, embodiments, features, and alternatives will now be further described, in relation to systems as well as methods.

The use of UV light, or other forms of non-ionizing radiation, effectively sterilize surfaces within process components. The process components may incorporate materials that are expressly incompatible with steam sterilization, which allows for the use of lower-cost materials, among many other benefits arising from UV sterilization.

Conventionally, sterile conditions are generated within bioprocess components using steam sterilization. Such bioprocess components are evacuated and subsequently pressurized with typically 121° C. saturated steam, usually for about 1 hour, or at 132° C. for about 5 minutes. The steam environment is maintained until the adventitious microorganisms are inactivated, at which point the steam is removed from the system and bioprocess operation can begin. This process of steam sterilization introduces significant costs to the system operator. First, a significant amount of energy is required to generate the steam, which is both expensive as well as highly carbon-intensive unless renewable energy is used to generate the steam, which adds more cost. Also, designing the bioreactor for steam compatibility introduces significant additional costs and complexities.

It is known that ultraviolet light can kill or deactivate living organisms. Ultraviolet radiation, mainly UV-C is one of the powerful agents that can alter the normal state of life by inducing a variety of mutagenic and cytotoxic DNA lesions such as cyclobutane-pyrimidine dimers, 6-4 photoproducts, and DNA strand breaks by interfering the genome integrity. See Rastogi et al., “Molecular Mechanisms of Ultraviolet Radiation-Induced DNA Damage and Repair”, Journal of Nucleic Acids, Volume 2010, Article ID 592980, Pages 1-32 (2010), which is hereby incorporated by reference.

In some variations, the disclosed process interconnection or media sterilization system is connected to a UV-sterilizable bioreactor system comprising:
(a) a chamber configured to carry out a reaction;
(b) a component configured for introducing a gas into the chamber; and
(c) one or more UV light sources configured to expose ultraviolet light to surfaces within the chamber.

In this specification, “UV-sterilizable” means that a system or component is capable of being sterilized by UV light, at least to some extent. In this specification, “sterilized”, “sterilize”, “sterilization”, and the like refer to the killing, deactivation, or removal of microorganism to various extents. Sterilization does not mean that absolutely all microorganisms have been killed, deactivated, or removed (which may be referred to as “aseptic”). Certain embodiments may provide aseptic conditions for the system or component being sterilized.

In some embodiments, a bioreactor is configured to carry out a bioreaction, such as fermentation or enzymatic conversion. In this specification, a “bioreactor” is a fermentor that utilizes at least one gas (typically air or oxygen) in the fermentation reaction(s). In other embodiments, a reactor chamber is configured to carry out an anaerobic bioreaction or a non-biological reaction that does not utilize microorganisms or enzymes.

A gas may be introduced to a reactor or bioreactor. The gas be a reactant, such as in aerobic or microaerobic fermentation, or may be a catalyst, promoter, reaction-rate moderator, or other reaction agent. The gas introduced to the process chamber may be air, oxygen, syngas, hydrogen, carbon monoxide, methane, natural gas, or a combination thereof. In typical embodiments, the gas is oxygen, air, oxygen-enriched air, or oxygen-depleted air.

An external oxygen concentration may be utilized to increase the oxygen content of air beyond the normal 21 vol % O₂ concentration. In various embodiments employing O₂ in fermentation, the O₂ concentration in the gas stream fed to the process chamber (e.g., through a sparger) is about, at least about, or at most about 1 vol %, 2 vol %, 5 vol %, 10 vol %, 15 vol %, 20 vol %, 21 vol %, 22 vol %, 25 vol %, 30 vol %, 40 vol %, 50 vol %, 60 vol %, 70 vol %, 80 vol %, 90 vol %, 95 vol %, or 100 vol %, including all intervening ranges.

In some embodiments, the component for introducing a gas is a gas sparger. A sparger may be defined as a component for introducing a gas into the liquid within a bioreactor. Three basic types of spargers are porous spargers, orifice spargers, and nozzle spargers. Spargers are tailored to introduce the desired gas in a controlled manner, resulting in mass transfer between the liquid phase and gas phase, while also introducing mechanical energy into the system.

In some embodiments, the component for introducing a gas is a membrane. These components serve to introduce the gaseous substrate via a semi-permeable membrane, allowing for selective mass transfer of desired components between a gaseous phase and a liquid phase. In some cases, this mass transfer occurs via diffusion within a polymeric matrix, while in other cases this mass transfer occurs via diffusion within pores that are within the membrane itself. In certain embodiments, the gaseous component being transported across the membrane is oxygen.

In some embodiments, the component for introducing a gas is a pipe or tube that is disposed in the volume of the bioreactor and that directly injects a gas into the liquid phase. The pipe or tube may have a single outlet, such as near the bottom of the chamber, near the top of the chamber, or anywhere else in the chamber. The pipe or tube may have multiple outlets, such as a perforated pipe with a plurality of holes, out of which a gas enters the liquid phase.

In some embodiments, the component for introducing a gas is a plate sparger. A plate sparger is typically located at the bottom of the chamber and has a plurality of holes, out of which a gas enters the liquid phase.

In some embodiments, the component for introducing a gas is UV-sterilizable.

The component (e.g., sparger) for introducing a gas may be UV-sterilizable by being exposed to a UV light source that is external to the component, and internal or external to the process chamber. Alternatively, or additionally, the component may be UV-sterilizable by incorporating a UV light source within the component itself.

In this specification, a “UV-transparent material” is not necessarily completely UV-transparent; some absorption of UV radiation may occur. In particular, “UV-transparent” means a sheet of material with 1-millimeter thickness absorbs less than 50%, preferably about 25% or less, more preferably about 10% or less, most preferably about 9%, 8%, 7%, 6%, 5%, 4%, 3%, 2%, 1%, or less, of incident (perpendicular) UV light at a wavelength of interest. The actual material or component need not be 1 millimeter; that thickness is only specified when measuring the UV transmission for purposes of this paragraph. If a wavelength range is used, the UV transmission is averaged over that range. UV transmission can be determined experimentally, for example, using a Perkin Elmer UV-Vis-IR spectrometer. It is noted that UV transmission generally can include regular UV transmission and diffuse UV transmission, both of which can contribute to total UV transmission.

A UV-transparent material may contain a UV-transparent polymer, a UV-transparent ceramic, a UV-transparent glass, or a combination thereof. A UV-transparent polymer may be selected from the group consisting of polyacrylates, silicones, fluoropolymers, and combinations thereof. Fluoropolymers may be selected from the group consisting of polyvinylidene fluoride, polytetrafluoroethylene, perfluoroalkoxy alkanes, and combinations thereof. A UV-transparent ceramic may be selected from the group consisting of quartz, fused silica, borosilicates, and combinations thereof. Silica or borosilicates may be doped to modify their UV transparency.

Within a particular system, the number of UV light sources may vary widely. In some systems, there is a single UV light source configured to expose ultraviolet light to all intended surfaces, including the process chamber, one or more process valves, and optionally bioreactor internal surfaces. In other embodiments, there are multiple UV light sources each configured to expose ultraviolet light to surfaces. The number of individual UV light sources can vary widely, such as 1, 2, 3, 4, 5, 6, 7, 8, 9, 10, 25, 50, 100, or more. In certain embodiments employing one or more micro-arrays of UV LED light sources, the number of individual UV light sources can be many hundreds or thousands.

In some embodiments, one or more UV light sources are situated within UV-transparent wells that are disposed within the process chamber. The UV-transparent wells may be fabricated from a UV-transparent material containing a UV-transparent polymer, a UV-transparent ceramic, a UV-transparent glass, or a combination thereof. A UV-transparent polymer may be selected from the group consisting of polyacrylates, silicones, fluoropolymers, and combinations thereof. Fluoropolymers may be selected from the group consisting of polyvinylidene fluoride, polytetrafluoroethylene, perfluoroalkoxy alkanes, and combinations thereof. A UV-transparent ceramic may be quartz. A UV-transparent glass may be fused silica, borosilicates, or a combination thereof. Silica or borosilicates may be doped to modify their UV transparency.

In some embodiments, at least some (such as at least one) of the one or more UV light sources are external to the process chamber and external to the process valves. In certain embodiments, all of the one or more UV light sources are external to the process chamber and external to the process valves.

Typically, the process chamber walls facing the inside of the process chamber are to be sterilized, but the outside chamber walls exposed to the environment do not need to be sterilized. If desired, the outside chamber walls may be sterilized as well.

In some embodiments, one or more UV light sources are configured to provide radiant UV flux to a corner of a process chamber, such as a corner defined by part of the chamber wall and an internal component (e.g., static mixer or agitator shaft). In certain embodiments, discrete UV LED fixtures are utilized for specific components that would otherwise be inaccessible to a primary UV light source, such as a UV mercury lamp.

In some embodiments, the process chamber and/or the process valves have walls fabricated from a metal, a metal alloy, a polymer, a ceramic, a composite material, glass, concrete, or a combination thereof. In certain embodiments, the metal is aluminum. In certain embodiments, the metal alloy is carbon steel or stainless steel. In certain embodiments, the polymer is selected from the group consisting of polyolefins, polyacrylates, polycarbonates, fluoropolymers, silicones, and combinations thereof. In certain embodiments, the composite material is a polymer reinforced with glass fibers. In certain embodiments, the polymer is selected from high-density polyethylene, polypropylene, polycarbonate, or a combination thereof. The polymer may be selected from the group consisting of poly(methyl methacrylate), polyvinylidene fluoride, hexafluoropropylene-tetrafluoroethylene copolymers (also known as fluorinated ethylene propylene), perfluoroether-tetrafluoroethylene copolymers, and combinations thereof.

In some embodiments, the process chamber and/or the process valves have UV-transparent walls containing a UV-transparent material. The UV-transparent material may contain a UV-transparent polymer, a UV-transparent ceramic (e.g., quartz), a UV-transparent glass (e.g., fused silica and/or borosilicates), or a combination thereof. A UV-transparent polymer may be selected from the group consisting of polyacrylates (e.g., poly(methyl methacrylate)), silicones, fluoropolymers, and combinations thereof. Fluoropolymers may be selected from the group consisting of polyvinylidene fluoride, hexafluoropropylene-tetrafluoroethylene copolymers (also known as fluorinated ethylene propylene), perfluoroether-tetrafluoroethylene copolymers, poly(ethene-co-tetrafluoroethene), or a combination thereof.

In certain embodiments, the process chamber and/or the process valves have UV-reflective chamber walls containing, or internally coated with, a UV-reflective material. The UV-reflective material may be selected from aluminum, stainless steel, polytetrafluoroethylene, or a combination thereof, for example.

The process chamber volume may vary widely for a given system, including laboratory scale, pilot scale, demonstration scale, and commercial scale. In various embodiments, the chamber volume is about, at least about, or at most about 10 mL, 50 mL, 100 mL, 250 mL, 1 L, 2 L, 5 L, 10 L, 25 L, 50 L, 100 L, 500 L, 1,000 L, or greater.

The process chamber geometry may also vary. A typical process chamber is cylindrical with rounded walls (circular with respect to the horizontal axis). A process chamber may have rounded walls, flat walls, or a combination thereof. Process chamber geometries may generally include cylindrical, tubular, conical, spherical, or rectangular. The aspect ratio of the process chamber may vary, such as tall (longer in the vertical dimension than the horizontal dimension) or short (longer in the horizontal dimension than the vertical dimension). The process chamber orientation may be vertical, horizontal, or slanted.

In some embodiments, the one or more UV light sources each have a UV wavelength selected from about 100 nm to about 400 nm. In certain embodiments, the UV wavelength is selected from about 220 nm to about 300 nm, for at least one of the one or more UV light sources, such as for all of the UV light sources. In various embodiments, the UV wavelength is about, at least about, or at most about 100, 110, 120, 125, 130, 140, 150, 160, 170, 180, 190, 200, 210, 220, 230, 240, 250, 260, 270, 280, 290, 300, 310, 320, 330, 340, 350, 360, 370, 380, 390, or 400 nm, including all intervening ranges, and inclusive of each 1-nanometer increment within each sub-range (e.g., including 227-304 nm, 188-242 nm, etc.). In some embodiments, such as (but not limited to) those utilizing pulsed light, multiple radiation wavelengths may be provided by the light source, some of which fall in the 100-400 nm range and some of which are higher than 400 nm, such as in the visible or infrared range in the electromagnetic spectrum. In certain embodiments, the one or more UV light sources each have a UV wavelength selected from 125 nm to 400 nm.

In certain embodiments, the one or more UV light sources each have a UV wavelength selected from UV-C wavelengths, which for purposes of this specification are 200-290 nm. In certain embodiments, the one or more UV light sources each have a UV wavelength selected from UV-B wavelengths, which for purposes of this specification are 291-320 nm. In certain embodiments, the one or more UV light sources each have a UV wavelength selected from UV-A wavelengths, which for purposes of this specification are 321-400 nm.

With respect to wavelength, while 100-400 nm is a preferred range of radiation wavelengths, it will be recognized that higher wavelengths can also be effective in certain embodiments, depending on the specific adventitious microorganism and the radiation parameters other than wavelength (e.g., time, light intensity, total power, or absorbed energy). Wavelengths in the visible band (about 400-700 nm) and/or infrared band (about 700 nm-1 mm) may be capable of inactivating adventitious microorganisms, such as by inducing DNA lesions.

The one or more UV light sources may each be selected from the group consisting of UV light-emitting diodes, UV mercury lamps, UV xenon lamps, and UV krypton lamps, for example. In certain embodiments, all UV light sources are UV light-emitting diodes (UV LEDs). A UV light source may be configured to generate pulsed UV light. For example, a pulsed-UV light source may be pulsed several times per second, with each pulse lasting between about 100 nanoseconds and 10 milliseconds.

In some embodiments, a bioreactor contains a biosensor situated within the bioreactor. The biosensor may be configured to detect or measure a parameter selected from the group consisting of pH, temperature, oxygen, carbon dioxide, foaming, mixing, cell density, feed-substrate concentration, reaction-intermediate concentration, and product concentration, for example. There may be multiple biosensors situated within the bioreactor. In some embodiments, the biosensor is configured to transmit a wireless signal to a computer for monitoring and control of the bioreaction system.

In some embodiments, the biosensor is UV-sterilizable. The biosensor may be configured with a UV optical waveguide for sterilizing the biosensor. The UV optical waveguide may be a UV optical fiber, for example.

In some embodiments, the biosensor is disposed within or through a probe port, wherein the probe port is UV-sterilizable. In some embodiments, the biosensor is contained in a UV-sterilizable housing that is situated within the bioreactor. The UV-sterilizable housing may contain a UV-transparent material, such as a UV-transparent polymer, a UV-transparent ceramic, a UV-transparent glass, or a combination thereof. A UV-transparent polymer may be selected from the group consisting of polyacrylates, silicones, fluoropolymers, and combinations thereof. An exemplary polyacrylate is poly(methyl methacrylate). Fluoropolymers may be selected from the group consisting of polyvinylidene fluoride, hexafluoropropylene-tetrafluoroethylene copolymers (also known as fluorinated ethylene propylene), perfluoroether-tetrafluoroethylene copolymers, poly(ethene-co-tetrafluoroethene), and combinations thereof. An exemplary UV-transparent ceramic is quartz. Exemplary UV-transparent glasses include fused silica, borosilicates, or a combination thereof. Silica or borosilicates may be doped to modify their UV transparency.

In some embodiments, aeration is accomplished using a gas-input component configured for introducing a gas into the bioreactor. The gas-input component may be a sparger, a membrane, or another means of effectively distributing a gas into the chamber. While it is possible to feed a gas through a port into the bioreactor, this may lead to highly non-uniform gas concentrations within the bioreactor. In some embodiments, a gas input is sterilized via UV sterilization, filtration, or a combination thereof. In some embodiments, the gas-input component may have UV-light sources mounted within the component, such that sterile conditions are generated within the interior of the gas-input component.

In some embodiments, an agitation component facilitates mixing of the internal constituents of the bioreactor. In some embodiments, the agitation component is a static mixer. In some embodiments, the agitation component is an impeller that achieves mixing by rotating around within the process chamber, transferring kinetic energy to the chamber contents (usually in turbulent flow, although can be laminar flow). The power to the impeller typically comes from an electric motor, i.e. powered by electricity, although in principle the impeller may be powered by magnetic induction or even compressed air. When an electrical motor is used, the motor is preferably mounted outside the process chamber, and the motor may be sealed using a mechanical seal, a lip seal, or a magnetic seal, any of which may be UV-sterilized.

A thermal-management component may introduce or remove thermal energy (heat) from the system, such as to or from the process chamber, or to or from the bioreactor. In some embodiments, heat transfer may occur through the walls, using a heat-transfer fluid, a steam jacket, heating coils, cooling coils, another thermal-management component, or a combination thereof. In some embodiments, internal baffles and/or internal coils are used to heat or cool the contents. An external flow loop may be used to heat or cool the contents.

Various components for sensing within the system may be used, such as bioreactor sensors or process-chamber sensors. In various embodiments, a bioreactor sensor detects or measures a bioreactor parameter selected from the group consisting of pH, temperature, dissolved oxygen, dissolved air, dissolved hydrogen, dissolved carbon monoxide, dissolved carbon dioxide, dissolved methane, foaming, mixing, cell density, feed-substrate concentration, reaction-intermediate concentration, product concentration, density, or weight, for example. The bioreactor sensor may measure various spectrophotometric characteristics. A bioreactor sensor may be configured with an optical microscope, camera, or IR scope to take a picture of the mass distribution and/or the heat distribution at certain locations in the bioreactor, such as near the sparger. In some embodiments, the bioreactor sensor is configured to transmit a wireless signal to a computer for monitoring and control of the system.

Sensors may also be used for measuring a property within a product stream or a property of a sample that is withdrawn from a vessel that is connected to the process chamber. Product sensors or sample sensors may detect or measure a parameter selected from the group consisting of pH, temperature, dissolved oxygen, dissolved air, dissolved hydrogen, dissolved carbon monoxide, dissolved carbon dioxide, dissolved methane, cell density, product concentration, density, or weight, for example. The measurement that is made with a sensor may be used to make process adjustments dynamically or in the future. The process adjustments may utilize well-known principles of process control, including proportional feedback control, proportional-integral-derivative (PID) feedback control, feedforward control, etc. A computer may be employed to automatically make adjustments to the process and system based on one or more measurements from sensors. Artificial intelligence may be used to automatically make adjustments to the process and system.

A step of UV sterilization may utilize a sterilization energy per unit total wettable area in the process chamber (and optionally, the wettable area in the process valves) of about 50, 100, 150, 200, 250, 300, 400, 500, 600, 700, 800, 900, 1000, 1500, 2000, 3000, 4000, or 5000 mJ/cm², or more, including all intervening ranges. The sterilization energy per unit total area is also known as the light intensity. The light intensity may be constant, or it may vary over time.

A total UV power output as power per unit total wettable area in the process chamber (and optionally, the wettable area in the process valves) may vary from about 0.5 mW/cm² to about 1000 mW/cm². The total UV power may be about, at least about, or at most about 0.5, 1, 2, 3, 4, 5, 10, 50, 100, 200, 300, 400, 500, 600, 700, 800, 900, 1000 mW/cm², including all intervening ranges. The total UV power output may be constant, or it may vary over time.

A step of UV sterilization may utilize a total UV power capacity from about 0.1 W/m³ to about 5000 W/m³. The total UV power capacity is calculated as input UV power divided by volume of the process chamber (and optionally, the wettable volume in the process valves). The total UV power capacity may be about, or at most about 5000, 4000, 3000, 2000, 1000, 500, 400, 300, 200, 100, 50, 40, 30, 25, 20, 15, 10, 9, 8, 7, 6, 5, 4, 3, 2, 1, 0.5, 0.4, 0.3, 0.2, or 0.1 W/m³, including all intervening ranges.

A step of UV sterilization may utilize a UV sterilization time from about 1 minute to about 24 hours. The UV sterilization time is the time that the UV light source is illuminated. In various embodiments, the UV sterilization time is about, at least about, or at most about 1 minute, 2 minutes, 3 minutes, 4 minutes, 5 minutes, 10 minutes, 15 minutes, 20 minutes, 25 minutes, 30 minutes, 45 minutes, 1 hour, 2 hours, 3 hours, 4 hours, 5 hours, 6 hours, 7 hours, 8 hours, 12 hours, 16 hours, 20 hours, or 24 hours, including all intervening ranges.

A step of UV sterilization may be conducted at a sterilization temperature from about 10° C. to about 95° C. In various embodiments, the sterilization temperature is about, at least about, or at most about 10° C., 15° C., 20° C., 25° C., 30° C., 35° C., 40° C., 45° C., 50° C., 55° C., 60° C., 65° C., 70° C., 75° C., 80° C., 85° C., 90° C., or 95° C., including any intervening range. In some embodiments, the sterilization temperature is not controlled and is at or about the ambient temperature, such as about 25° C. In other embodiments, it is desirable to increase the temperature to assist in the sterilization.

In some methods, UV sterilization is conducted in an atmosphere that consists essentially of air, which may be dry air or humid air. In typical methods, UV sterilization is conducted in an atmosphere that does not contain steam, although water vapor may be present, due to humidity in air, for example. In some methods, UV sterilization is conducted in an inert-gas atmosphere, using N₂, Ar, or CO₂, for example.

In certain methods, UV sterilization is conducted in an atmosphere that contains a sterilization-enhancing vapor or gas, such as ethylene oxide, chlorine dioxide, hydrogen peroxide, ozone, formaldehyde, peracetic acid, or glutaraldehyde. Ozone may be generated in situ from photolysis of oxygen (e.g., from air), creating O₃ from O₂, such as when one or more UV wavelengths in the 160-240 nm range are used for the UV sterilization.

In various methods, UV sterilization is effective to reach a 4-log reduction in adventitious microorganisms present prior to the UV sterilization. In this specification, “log” refers to the common base-10 logarithm. Thus, a 4-log reduction is a 99.99% reduction in population of adventitious microorganisms because the fraction of adventitious microorganisms remaining is 10⁻⁴, and log(10⁻⁴)=−4. In some methods, UV sterilization is effective to reach a 6-log reduction in adventitious microorganisms present prior to the UV sterilization. In certain methods, UV sterilization is effective to reach a 8-log reduction in adventitious microorganisms present prior to the UV sterilization. In specific methods, UV sterilization is effective to reach a 10-log reduction in adventitious microorganisms present prior to the UV sterilization. In various embodiments, UV sterilization is effective to reach a reduction in adventitious microorganisms, present initially prior to such UV sterilization, that is about, or at least about, 3, 4, 5, 6, 7, 8, 9, 10, 11, or 12 logs. In certain embodiments, UV sterilization is effective to reach a complete elimination of adventitious microorganisms, present initially prior to the UV sterilization, i.e. 100% reduction (mathematically, ∞-log reduction) which may be referred to as achieving aseptic conditions. Generally speaking, different microorganisms have different vulnerabilities to UV light. When there are multiple species of adventitious microorganisms present, the kill rates of the different species may differ.

In bioreactor facilities, adventitious microorganisms can include spore-forming gram-positive or gram-negative rods; non-spore-forming bacteria; fungal contaminations; or cocci, for example. The microorganism for the desired fermentation may or may not be more UV-tolerant than adventitious microorganisms. If the adventitious microorganisms are especially UV-tolerant and survive the UV sterilization, this may pose no problem for the eventual fermentation in the bioreactor.

A typical bioprocess involves fermentation. To carry out a desired fermentation, there will generally be several inputs such as, but not limited to, media, a feedstock acting as a carbon source, acid input, base input, anti-foaming agent, buffer, vitamins, gaseous input, or any bolus of materials intended to influence the biochemical process. The fermentation may include introducing gas to the reaction chamber. The gas may be air, oxygen, syngas, hydrogen, carbon monoxide, methane, natural gas, or a combination thereof, for example. The gas may be a mixture of O₂ and N₂ in various concentrations of O₂, above or below 21 vol % O₂. The gas may be a reactant, such as in aerobic or microaerobic fermentation, or may be a catalyst, promoter, reaction-rate moderator, or other reaction agent. The gas may be first sterilized prior to being fed to the bioreactor, via filtration, exposure to UV light, or a combination thereof. This specification also hereby incorporates by reference Stanbury et al., Principles of Fermentation Technology, 3^{rd} Edition, Elsevier, 2017.

Following production of a fermentation product, the product will typically be present in a dilute solution or broth. Product recovery may be performed using known techniques, such as (but not limited to) evaporation, distillation, centrifuge, liquid-liquid extraction, to generate a concentrated form of a desired product. In certain embodiments, no product concentration is necessary because the as-is fermentation broth is the product which may be stored, shipped, or used elsewhere at a plant site. In some embodiments, one or more materials recovered from a dilute solution or broth is recovered and recycled for reuse at a plant site or an adjacent site, for example. For example, water may be recovered and reused, to improve the water balance. Vitamins and minerals may be recovered and reused in another fermentation. Any recovered materials may be subjected to UV sterilization prior to reuse, or UV sterilization at the point of re-entry into a process, such as via UV-sterilized filters. In some embodiments, biological material is inactivated upon removal from the bioreactor system via UV sterilization.

Generally speaking, the principles of the invention can be applied to a wide variety of commercial bioprocesses and products, including (but not limited to) industrial chemicals, biochemicals, biofuels, pharmaceuticals, nutraceuticals, vitamins, food ingredients, protein products, enzymes, and cells.

In this detailed description, reference has been made to multiple embodiments which show by way of illustration specific exemplary embodiments of the invention. These embodiments are described in sufficient detail to enable those skilled in the art to practice the invention, and it is to be understood that modifications to the various disclosed embodiments may be made by a skilled artisan.

Where methods and steps described above indicate certain events occurring in certain order, those of ordinary skill in the art will recognize that the ordering of certain steps may be modified and that such modifications are in accordance with the variations of the invention. Additionally, certain steps may be performed concurrently in a parallel process when possible, as well as performed sequentially.

All publications, patents, and patent applications cited in this specification are herein incorporated by reference in their entirety as if each publication, patent, or patent application were specifically and individually put forth herein.

The embodiments and variations described above should provide an indication of the utility and versatility of the present invention. Other embodiments that do not provide all of the features and advantages set forth herein may also be utilized, without departing from the spirit and scope of the present invention. Such modifications and variations are considered to be within the scope of the invention defined by the claims.

What is claimed is:

1. A process interconnection comprising:
(a) a first process valve comprising a first outer port and a first inner port;
(b) a second process valve comprising a second outer port and a second inner port, wherein said second process valve is in flow communication with said first process valve;
(c) a process chamber interposed between said first inner port and said second inner port, wherein said process chamber is in flow communication with said first process valve and with said second process valve, and wherein said process chamber has internal chamber surfaces; and
(d) one or more UV light sources configured to (i) expose ultraviolet light to at least 99% of chamber internal surface area defined by total surface area of said internal chamber surfaces and (ii) expose said ultraviolet light to at least 99% of valve internal surface area defined by total surface area of all internal, wettable valve surfaces within said first process valve and said second process valve.

2. The process interconnection of claim 1, wherein said one or more UV light sources are configured to expose said ultraviolet light to 100% of said chamber internal surface area and to 100% of said valve internal surface area.

3. The process interconnection of claim 1, wherein said first process valve, said second process valve, and said process chamber are fabricated from materials that are substantially chemically inert to said ultraviolet light.

4. The process interconnection of claim 1, wherein said process interconnection further comprises a third process valve having a third outer port and a third inner port, wherein said third process valve is in flow communication with said process chamber, said first process valve, and said second process valve.

5. The process interconnection of claim 1, wherein said process interconnection further comprises a third process valve having a third outer port and a third inner port, wherein said process interconnection further comprises a fourth process valve having a fourth outer port and a fourth inner port, wherein said third process valve is in flow communication with said process chamber, wherein said fourth process valve is in flow communication with said process chamber, and wherein said third process valve is in flow communication with said fourth process valve.

6. The process interconnection of claim 1, wherein said first inner port is connected to said process chamber using a first sanitary connection, and wherein said second inner port is connected to said process chamber using a second sanitary connection.

7. The process interconnection of claim 1, wherein said first inner port is connected to said process chamber using a first reversible sanitary connection, and wherein said second inner port is connected to said process chamber using a second reversible sanitary connection.

8. The process interconnection of claim 1, wherein said one or more UV light sources are positioned externally to said process chamber, said first process valve, and said second process valve, wherein said process chamber is configured with chamber walls that are substantially transparent or translucent to said ultraviolet light, and optionally wherein said first process valve and said second process valve are configured with valve walls that are substantially transparent or translucent to said ultraviolet light.

9. The process interconnection of claim 1, wherein said one or more UV light sources are positioned internally within said process chamber, and optionally wherein said one or more UV light sources are located inside a sleeve that is positioned inside said process chamber, wherein said sleeve is substantially transparent or translucent to said ultraviolet light.

10. The process interconnection of claim 1, wherein said process chamber is a vessel configured to contain a bioprocessing material.

11. A system for bioprocessing, said system comprising:
(a) a bioreactor comprising a bioreactor chamber configured to carry out a reaction;
(b) a bioreactor port configured for introducing bioreaction media to said bioreactor chamber;
(c) a first process valve comprising a first outer port and a first inner port, wherein said first process valve is in flow communication with said bioreactor port;
(d) a process chamber interposed between said first inner port and said bioreactor port, wherein said process chamber is in flow communication with said first process valve and with said bioreactor port, and wherein said process chamber has internal chamber surfaces; and
(e) one or more UV light sources configured to (i) expose ultraviolet light to at least 99% of chamber internal surface area defined by total surface area of said internal chamber surfaces and (ii) expose said ultraviolet light to at least 99% of first-valve internal surface area defined by total surface area of all internal, wettable valve surfaces within said first process valve.

12. The system of claim 11, wherein said one or more UV light sources are configured to expose said ultraviolet light to 100% of said chamber internal surface area and to 100% of said first-valve internal surface area.

13. The system of claim 11, wherein said first process valve and said process chamber are fabricated from materials that are substantially chemically inert to said ultraviolet light.

14. The system of claim 11, wherein said bioreactor port is a sanitary bioreactor port.

15. The system of claim 11, wherein said system further comprises a second process valve interposed between said process chamber and said bioreactor port, wherein said second process valve comprises a second outer port and a second inner port, wherein said second process valve is in flow communication with said first process valve and with said bioreactor chamber, and wherein said one or more UV light sources are configured to expose said ultraviolet light to at least 99% of second-valve internal surface area defined by total surface area of all internal, wettable valve surfaces within said second process valve.

16. The system of claim 15, wherein said system further comprises a third process valve having a third outer port and a third inner port, wherein said third process valve is in flow communication with said process chamber, said first process valve, and said second process valve.

17. The system of claim 11, wherein said bioreactor comprises a component configured for introducing a gas into said bioreactor chamber.

18. The system of claim 11, wherein said bioreactor comprises one or more bioreactor UV light sources configured to expose additional ultraviolet light to surfaces within said bioreactor chamber.

19. The system of claim 11, wherein said one or more UV light sources are positioned externally to said process chamber and said first process valve, wherein said process chamber is configured with chamber walls that are substantially transparent or translucent to said ultraviolet light, and optionally wherein said first process valve is configured with valve walls that are substantially transparent or translucent to said ultraviolet light.

20. The system of claim 11, wherein said one or more UV light sources are positioned internally within said process chamber.
